(19) United States
(12) Patent Application Publication
(10) Pub. No.: US 2025/0250284 A1
(43) Pub. Date: Aug. 7, 2025

(54) MACROCYCLE COMPOUNDS USEFUL AS KRAS INHIBITORS

(71) Applicant: Hoffmann-La Roche Inc., Little Falls, NJ (US)

(72) Inventors: Jianguo CHEN, Shanghai (CN); Haixia LIU, Shanghai (CN); Hong SHEN, Shanghai (CN); Weixing ZHANG, Shanghai (CN); Dan ZHAO, Shanghai (CN); Wei ZHU, Shanghai (CN)

(73) Assignee: Hoffmann-La Roche Inc., Little Falls, NJ (US)

(21) Appl. No.: 19/008,878

(22) Filed: Jan. 3, 2025

Related U.S. Application Data
(63) Continuation of application No. PCT/EP2023/068641, filed on Jul. 6, 2023.

(30) Foreign Application Priority Data
Jul. 8, 2022 (WO) PCT/CN2022/104656
Jan. 5, 2023 (WO) PCT/CN2023/070764

(51) Int. Cl. C07D 515/22; A61K 31/504; A61K 31/5377

(52) U.S. Cl. CPC C07D 515/22; A61K 31/504; A61K 31/5377

(57) ABSTRACT

The present invention relates to compounds of formula (I),

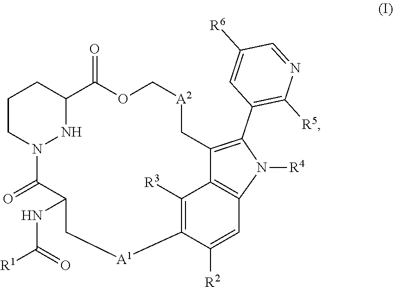

wherein R¹ to R⁶, A¹ and A² are as described herein, and their pharmaceutically acceptable salt thereof, and compositions including the compounds and methods of using the compounds.

### CROSS REFERENCE TO RELATED APPLICATIONS

This application is a continuation of International Application No. PCT/EP2023/068641, filed Jul. 6, 2023, which claims priority to CN Application No. PCT/CN2023/070764, filed Jan. 5, 2023, and CN Application No. PCT/CN2022/104656, filed Jul. 8, 2022, the disclosures of each of which are incorporated herein by reference in their entirety.

The present invention relates to organic compounds useful for therapy and/or prophylaxis in a mammal, and in particular to inhibition of KRAS mutant useful for treating cancers.

### FIELD OF THE INVENTION

RAS is one of the most well-known proto-oncogenes. Approximately 30% of human cancers contain mutations in three most notable members, KRAS, HRAS, and NRAS, making them the most prevalent oncogenic drivers. KRAS mutations are generally associated with poor prognosis especially in colorectal cancer, pancreatic cancer, lung cancers. As the most frequently mutated RAS isoform. KRAS has been intensively studied in the past years. Among the most commonly occurring KRAS alleles (including G12D, G12V, G12C, G13D, G12R, G12A, G12S, Q61H, etc), G12C, G12D, G12V represent more than half of all K-RAS-driven cancers across colorectal cancer (CRC), pancreatic ductal adenocarcinoma (PDAC), lung adenocarcinoma (LUAD). Of note, KRAS wild-type amplifications are also found in around 7% of all KRAS-altered cancers (ovarian, esophagogastric, uterine), ranking among the top alterations.

All RAS proteins belong to a protein family of small GTPases that hydrolyze GTP to GDP. KRAS is structurally divided into an effector binding lobe followed by the allosteric lobe and a carboxy-terminal region that is responsible for membrane anchoring. The effector lobe comprises the P-loop, switch I, and switch II regions. The switch I/II loops play a critical role in KRAS downstream signaling through mediating protein-protein interactions with effector proteins that include RAF in the mitogen-activated protein kinase (MAPK) pathway or PI3K in the phosphatidylinositol 3-kinase (PI3K)/protein kinase B (AKT) pathway.

KRAS protein switches between an inactive to an active form via binding to GTP and GDP, respectively. Under physiological conditions, the transition between these two states is regulated by guanine nucleotide exchange factors (GEFs), such as Son Of Sevenless Homolog 1 (SOS1), or GTPase-activating proteins (GAPs) that involve catalyzing the exchange of GDP for GTP, potentiating intrinsic GTPase activity or accelerating RAS-mediated GTP hydrolysis. In response to extracellular stimuli, the inactive RAS-GDP is converted to active RAS-GTP which directly binds to RAF RAS binding domains (RAF^{RBD}), recruiting RAF kinase family from cytoplasm to membranes, where they dimerize and become active. The activated RAF subsequently carries out a chain of phosphorylation reactions to its downstream Mitogen-activated protein kinase (MEK) and extracellular signal-regulated kinase (ERK), and propagates the growth signal. Of the RAF family of protein kinases (three known isoforms ARAF, BRAF, CRAF/RAF1), BRAF is most frequently mutated and remains the most potent activator of MEK. Despite that individual RAS and RAF family members revealed distinct binding preferences, all RAFs possess the conserved RBD for forward transmission of MAPK singnaling, frequently used for characterize KRAS inhibition (e.g. KRAS-BRAF^{RBD} herein). For KRAS, mutations at positions 12, 13, 61, and 146 lead to a shift toward the active KRAS form through impairing nucleotide hydrolysis or activating nucleotide exchange, leading to hyper-activation of the MAPK pathway that results in tumorigenesis.

Despite its well-recognized importance in cancer malignancy, continuous efforts in the past failed to develop approved therapies for KRAS mutant cancer until recently, the first selective drug AMG510 has fast approval as second line treatment in KRAS G12C driven non-small cell lung cancer (NSCLC). Nevertheless, the clinical acquired resistance to KRAS G12C inhibitors emerge rigorously with disease progresses after around 6 month of treatment. All of the mutations converge to reactivate RAS-MAPK signaling, with secondary RAS mutants at oncogenic hotspots (e.g. G12/G13/Q61) and within the switch II pocket (e.g. H95, R68, and Y96) have been observed; moreover, over 85% of all KRAS-mutated or wild-type amplified driven cancers still lack novel agents. Altogether, both the myriad of escape mechanism and various oncogenic alleles, highlight the urgent medical need for additional KRAS therapies. As such, we invented oral compounds that target and inhibit KRAS alleles for the treatment of KRAS mutant driven cancers.

### SUMMARY OF THE INVENTION

The present invention relates to novel compounds of formula (I),

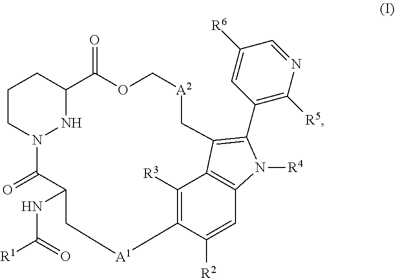

wherein
R¹ is 3-oxabicyclo[3.1.0]hexanyl, C₁₋₆alkylC₃₋₇cycloalkyl, cyanoC₃₋₇cycloalkyl or haloC₁₋₆alkylC₃₋₇cycloalkyl;
R² is H or halogen;
R³ is H or halogen;
R⁴ is C₁₋₆alkyl or haloC₁₋₆alkyl;
R⁵ is C₁₋₆alkoxyC₁₋₆alkyl;
R⁶ is morpholinyl, (haloC₁₋₆alkyl)piperazinyl or C₁₋₆alkylpiperazinyl;
A¹ is thiazolylene;
A² is C₁₋₆alkylene;
with the proviso that R² and R³ are not H simultaneously;
or a pharmaceutically acceptable salt thereof.

The invention also relates to their manufacture, medicaments based on a compound in accordance with the invention and their production as well as the use of compounds of formula (I) or (Ia) thereof as inhibitor of KRAS.

The compounds of formula (I) or (Ia) show good KRAS inhibition for G12C and G12V. In another embodiment, the compounds of this invention showed superior cancer cell inhibition and human hepatocyte stability. In addition, the compounds of formula (I) or (Ia) also show good or improved cytotoxicity and solubility profiles. Furthermore, the compound of current invention had good pharmacokinetic properties comparing with the reference compounds

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “C₁₋₆alkoxy” denotes C₁₋₆alkyl-O—.

The term “C₁₋₆alkylene” denotes a linear or branched saturated divalent hydrocarbon group of 1 to 6 carbon atoms or a divalent branched saturated divalent hydrocarbon group of 3 to 6 carbon atoms. Examples of C₁₋₆alkylene groups include methylene, ethylene, propylene, 2-methylpropylene, butylene, 2-ethylbutylene, pentylene, hexylene.

The term “halogen” and “halo” are used interchangeably herein and denote fluoro, chloro, bromo, or iodo.

The term “haloC₁₋₆alkyl” denotes a C₁₋₆alkyl group wherein at least one of the hydrogen atoms of the C₁₋₆alkyl group has been replaced by same or different halogen atoms, particularly fluoro atoms. Examples of haloalkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, or trifluoromethyl.

The term “C₃₋₇cycloalkyl” denotes a monovalent saturated monocyclic or bicyclic hydrocarbon group of 3 to 7 ring carbon atoms. Bicyclic means consisting of two saturated carbocycles having one or more carbon atoms in common. Examples for monocyclic cycloalkyl are cyclopropyl, cyclobutyl, cyclopentyl, cyclohexyl or cycloheptyl. Examples for bicyclic cycloalkyl are bicyclo[1.1.0]butyl, bicyclo[2.2.1]heptanyl, bicyclo[1.1.1]pentanyl, or bicyclo[2.2.2]octanyl.

The term “thiazolylene” denotes a divalent thiazolyl group.

The term “oxo” denotes a divalent oxygen atom ═O.

The term “dimethylmethylene” denotes

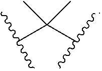 [SMILES: *C(*)(C)C]

The term “protecting group” denotes the group which selectively blocks a reactive site in a multifunctional compound such that a chemical reaction can be carried out selectively at another unprotected reactive site in the meaning conventionally associated with it in synthetic chemistry. Protecting groups can be removed at the appropriate point. Exemplary protecting groups are amino-protecting groups, carboxy-protecting groups or hydroxy-protecting groups.

The skilled of the art would understand that the following structures of compounds of formula (I) and (I′) are equal especially for the chiral centers:

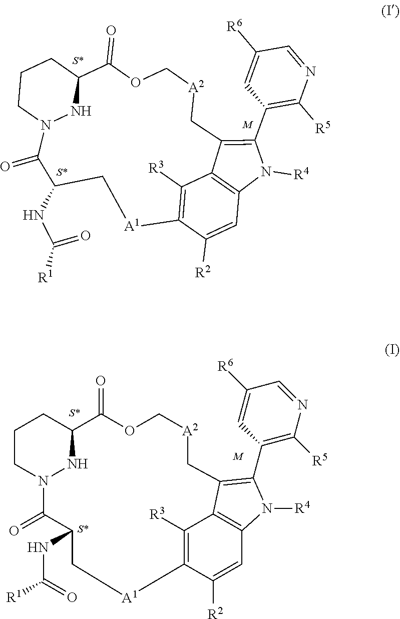

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

### Inhibitor of KRAS

The present invention relates to (i) a compound of formula (I),

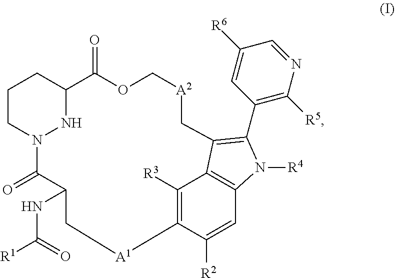

wherein
R¹ is 3-oxabicyclo[3.1.0]hexanyl, C₁₋₆alkylC₃₋₇cycloalkyl, cyanoC₃₋₇cycloalkyl or haloC₁₋₆alkylC₃₋₇cycloalkyl;
R² is H or halogen;
R³ is H or halogen;
R⁴ is C₁₋₆alkyl or haloC₁₋₆alkyl;
R⁵ is C₁₋₆alkoxyC₁₋₆alkyl;
R⁶ is morpholinyl, (haloC₁₋₆alkyl)piperazinyl or C₁₋₆alkylpiperazinyl;
A¹ is thiazolylene;
A² is C₁₋₆alkylene;
with the proviso that R² and R³ are not H simultaneously;
or a pharmaceutically acceptable salt thereof.

Another embodiment of present invention is (ii) a compound of formula (Ia),

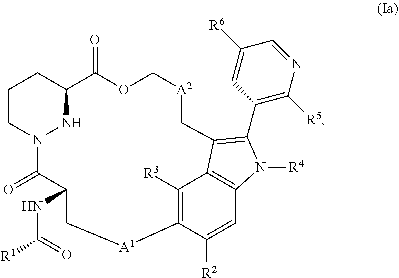

wherein
R¹ is 3-oxabicyclo[3.1.0]hexanyl, C₁₋₆alkylC₃₋₇cycloalkyl, cyanoC₃₋₇cycloalkyl or haloC₁₋₆alkylC₃₋₇cycloalkyl;
R² is H or halogen;
R³ is H or halogen;
R⁴ is C₁₋₆alkyl or haloC₁₋₆alkyl;
R⁵ is C₁₋₆alkoxyC₁₋₆alkyl;
R⁶ is morpholinyl, (haloC₁₋₆alkyl)piperazinyl or C₁₋₆alkylpiperazinyl;
A¹ is thiazolylene;
A² is C₁₋₆alkylene;
with the proviso that R² and R³ are not H simultaneously;
or a pharmaceutically acceptable salt thereof.

A further embodiment of present invention is (iii) a compound of formula (I) or (Ia) according to (i) or (ii), or a pharmaceutically acceptable salt thereof, wherein R¹ is C₁₋₆alkylC₃₋₇cycloalkyl or haloC₁₋₆alkylC₃₋₇cycloalkyl.

A further embodiment of present invention is (iv) a compound of formula (I) or (Ia), according to any one of (i) to (iii), or a pharmaceutically acceptable salt thereof, wherein R¹ is methylcyclopropyl or (difluoromethyl)cyclopropyl.

A further embodiment of present invention is (v) a compound of formula (I) or (Ia) according to any one of (i) to (iv), wherein R² is H or fluoro.

A further embodiment of present invention is (vi) a compound of formula (I) or (Ia), or a pharmaceutically acceptable salt thereof, according to any one of (i) to (v), wherein R³ is H or fluoro.

A further embodiment of present invention is (vii) a compound of formula (I) or (Ia), or a pharmaceutically acceptable salt thereof, according to any one of (i) to (vi), wherein R⁴ is ethyl or 2,2,2-trifluoroethyl.

A further embodiment of present invention is (viii) a compound of formula (I) or (Ia), or a pharmaceutically acceptable salt thereof, according to any one of (i) to (vii), wherein R⁵ is 1-methoxyethyl.

A further embodiment of present invention is (ix) a compound of formula (I) or (Ia), or a pharmaceutically acceptable salt thereof, according to any one of (i) to (viii), wherein R⁶ is morpholinyl or C₁₋₆alkylpiperazinyl.

A further embodiment of present invention is (x) a compound of formula (I) or (Ia), or a pharmaceutically acceptable salt thereof, according to any one of (i) to (ix), wherein R⁶ is morpholinyl or 4-methylpiperazin-1-yl.

A further embodiment of present invention is (xi) a compound of formula (I) or (Ia), or a pharmaceutically acceptable salt thereof, according to any one of (i) to (x), wherein A¹ is

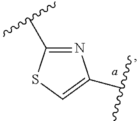 [SMILES: *c1csc(*)n1]

wherein bond “a” connects to indole ring.

A further embodiment of present invention is (xii) a compound of formula (I) or (Ia), or a pharmaceutically acceptable salt thereof, according to any one of (i) to (xi), wherein A² is dimethylmethylene.

Another embodiment of present invention is (xiii) a compound of formula (I) or (Ia), according to (i) or (ii), wherein
R¹ is C₁₋₆alkylC₃₋₇cycloalkyl or haloC₁₋₆alkylC₃₋₇cycloalkyl;
R² is H or halogen;
R³ is H or halogen;
R⁴ is C₁₋₆alkyl or haloC₁₋₆alkyl;
R⁵ is C₁₋₆alkoxyC₁₋₆alkyl;
R⁶ is morpholinyl or C₁₋₆alkylpiperazinyl;
A¹ is

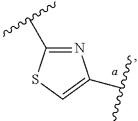 [SMILES: *c1csc(*)n1]

wherein bond “a” connects to indole ring;
A² is C₁₋₆alkylene;
with the proviso that R² and R³ are not H simultaneously;
or a pharmaceutically acceptable salt thereof.

Another embodiment of present invention is (xiv) a compound of formula (I) or (Ia), according to (xiii), wherein
R¹ is 2-methylcyclopropyl or 2-(difluoromethyl)cyclopropyl;
R² is H or fluoro;
R³ is H or fluoro;
R⁴ is ethyl or 2,2,2-trifluoroethyl;
R⁵ is (1S)-1-methoxyethyl;
R⁶ is morpholinyl or 4-methylpiperazin-1-yl;
A¹ is

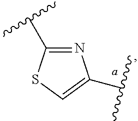 [SMILES: *c1csc(*)n1]

wherein bond “a” connects to indole ring;
A² is dimethylmethylene;
with the proviso that R² and R³ are not H simultaneously;
or a pharmaceutically acceptable salt thereof.

Another embodiment of present invention is (xv) a compound of formula (I) or (Ia) selected from the following;
(1S,2S)—N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide;
(1R,5S)—N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-oxabicyclo[3.1.0]hexane-6-carboxamide;
(1S,2S)-2-(difluoromethyl)-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]cyclopropanecarboxamide;
(1S,2S)—N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide;
(1S,2S)-2-cyano-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]cyclopropanecarboxamide;
(1R,2R)-2-cyano-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]cyclopropanecarboxamide;
(1S,2S)—N-[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide;
(1S,2S)—N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.11^{2,5}.1^{9,13}.0^{22,26}]octacosa-1(25),2,5(28),19,22(26),23-hexaen-7-yl]-2-methyl-cyclopropanecarboxamide;
(1S,2S)—N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide;
(1S,2S)—N-[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide;
(1S,2S)-2-(difluoromethyl)-N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]cyclopropanecarboxamide; and
(1S,2S)—N-[(7S,13S)-24-fluoro-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide;
or a pharmaceutically acceptable salt thereof.

Another embodiment of present invention is related to (xvi) a process for the preparation of a compound according to any one of (i) to (xv) comprising the following step:
a) coupling reaction between compound of formula (II),

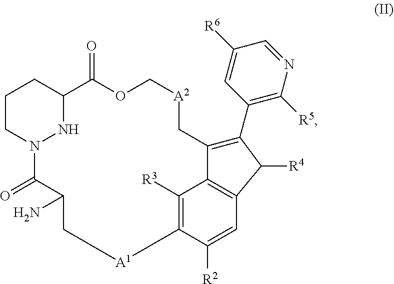

and acid (III),

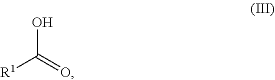

in the presence of a coupling reagent and a base to form the compound of formula (I);
wherein R¹, R², R³, R⁴, R⁵, R⁶, R⁷, A¹ and A² are defined as in any one of (i) to (xiv); the coupling reagent is T₃P, HATU, PyBOP or EDCI/HOBt; the base is TEA, DIEPA or DMAP.

Another embodiment of present invention is (xvii) a compound or pharmaceutically acceptable salt according to any one of (i) to (xv) for use as therapeutically active substance.

Another embodiment of present invention is (xviii) a pharmaceutical composition comprising a compound in accordance with any one of (i) to (xv) and a pharmaceutically acceptable excipient.

Another embodiment of present invention is (xix) the use of a compound according to any one of (i) to (xv) for treating a KRAS G12C protein-related disease.

Another embodiment of present invention is (xx) the use of a compound according to any one of (i) to (xv) for treating a KRAS G12C, G12D and G12V protein-related disease.

Another embodiment of present invention is (xxi) the use of a compound according to any one of (i) to (xv) for inhibiting RAS interaction with downstream effectors, wherein the downstream effectors are RAF and PI3K.

Another embodiment of present invention is (xxii) the use of a compound according to any one of (i) to (xv) for inhibiting the propagating oncogenic MAPK and PI3K signaling.

Another embodiment of present invention is (xxiii) the use of a compound according to any one of (i) to (xv) for the treatment or prophylaxis of KRAS mutation driven cancers, wherein the cancer is selected from pancreatic cancer, colorectal cancer, lung cancer, esophageal cancer, gallbladder cancer, melanoma ovarian cancer and endometrial cancer.

Another embodiment of present invention is (xxiv) the use of a compound according to any one of (i) to (xv) for the treatment or prophylaxis of KRAS mutation driven cancers, wherein the cancer is selected from pancreatic adenocarcinoma, colorectal cancer and non-small cell lung cancer.

Another embodiment of present invention is (xxV) a compound or pharmaceutically acceptable salt according to any one of (i) to (xv) for the treatment or prophylaxis of KRAS mutation driven cancers, wherein the cancer is selected from pancreatic adenocarcinoma, colorectal cancer and non-small cell lung cancer.

Another embodiment of present invention is (xxvi) the use of a compound according to any one of (i) to (xv) for the preparation of a medicament for the treatment or prophylaxis of KRAS mutation driven cancers, wherein the cancer is selected from pancreatic adenocarcinoma, colorectal cancer and non-small cell lung cancer.

Another embodiment of present invention is (xxvii) a method for the treatment or prophylaxis of KRAS mutation driven cancers, wherein the cancer is selected from pancreatic adenocarcinoma, colorectal cancer and non-small cell lung cancer, which method comprises administering a therapeutically effective amount of a compound as defined in any one of (i) to (XV).

Another embodiment of present invention is (xxviii) a compound or pharmaceutically acceptable salt according to any one of (i) to (xv), when manufactured according to a process of (xvi).

### Pharmaceutical Compositions and Administration

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit mutant RAS (e.g. KRAS G12C) interaction with RAF, blocking the oncogenic MAPK signaling. For example, such amount may be below the amount that is toxic to normal cells, or the mammal as a whole.

In one example, the pharmaceutically effective amount of the compound of the invention administered parenterally per dose will be in the range of about 0.1 to 1000 mg/kg, alternatively about 0.1 to 1000 mg/kg of patient body weight per day, with the typical initial range of compound used being 0.3 to 15 mg/kg/day. In another embodiment, oral unit dosage forms, such as tablets and capsules, preferably contain from about 1 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel. Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 1 to 1000 mg of the compound of the invention compounded with about 1 to 1000 mg anhydrous lactose, about 1 to 1000 mg sodium croscarmellose, about 1 to 1000 mg polyvinylpyrrolidone (PVP) K30, and about 1 to 1000 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a stereoisomer or pharmaceutically acceptable salt thereof. In a further embodiment includes a pharmaceutical composition comprising a compound of formula (I), or a stereoisomer or pharmaceutically acceptable salt thereof, together with a pharmaceutically acceptable carrier or excipient.

Another embodiment includes a pharmaceutical composition comprising a compound of formula (I) for use in the treatment of mutant KRAS-driven cancers. Another embodiment includes a pharmaceutical composition comprising a compound of Formula (I) for use in the treatment of mutant KRAS-driven cancers.

The following composition A and B illustrate typical compositions of the present invention, but serve merely as representative thereof.

### Composition A

A compound of the present invention can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Composition B

A compound of the present invention can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Indications and Methods of Treatment

The compounds of the invention induce a new binding pocket in KRAS by driving formation of a high affinity tri-complex between KRAS protein and the widely expressed cyclophilin A (CYPA), which inhibit KRAS interaction with downstream effectors, such as RAF and PI3K. Accordingly, the compounds of the invention are useful for inhibiting the propagating oncogenic MAPK and PI3K signaling, reducing cell proliferation, in particular cancer cells. Compounds of the invention are useful for termination of RAS signaling in cells that express RAS mutant, e.g. KRAS mutation driven pancreatic cancer, colorectal cancer, lung cancer, esophageal cancer, gallbladder cancer, melanoma ovarian cancer, endometrial cancer, etc. Alternatively, compounds of the invention are useful for termination of RAS signaling in malignant solid tumor where the oncogenic role of KRAS mutation is reinforced by dysregulation or mutation of effector pathways as MAPK, PI3K-AKT-mTOR (Mammalian target of rapamycin) driven signaling, for targeted therapy in pancreatic adenocarcinoma, colorectal cancer, non-small cell lung cancer, etc.

Another embodiment includes a method of treating or preventing cancer in a mammal in need of such treatment, wherein the method comprises administering to said mammal a therapeutically effective amount of a compound of formula (I), a stereoisomer, tautomer, prodrug or pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, R¹ to R⁶, A¹ and A² are as defined above unless otherwise indicated. Furthermore, and unless explicitly otherwise stated, all reactions, reaction conditions, abbreviations and symbols have the meanings well known to a person of ordinary skill in organic chemistry.

General synthetic routes for preparing the compound of formula (I) are shown below.

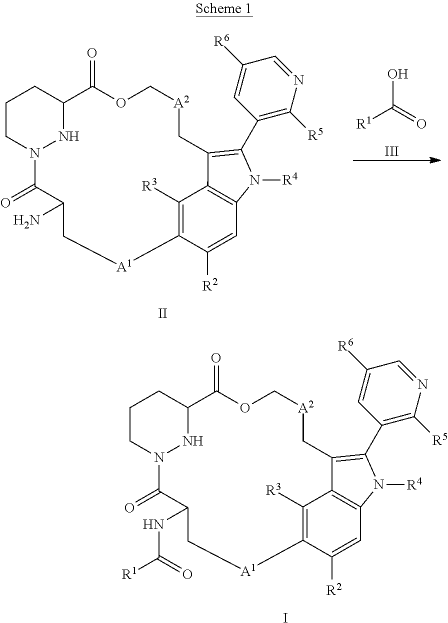

Compound of formula II was synthesized according to the procedure described in Intermediate A to I. Compound of formula (I) can be obtained by a coupling reaction between acid (III) and compound of formula (II) with coupling reagent(s), such as T₃P, HATU, PyBOP and EDCI/HOBt, in the presence of a base, such as TEA, DIEPA and DMAP.

Compounds of this invention can be obtained as mixtures of diastereomers or enantiomers, which can be separated by methods well known in the art, e.g. (chiral) HPLC or SFC. In another embodiment, compound of formula (I) can be obtained according to above scheme by using corresponding chiral starting materials.

This invention also relates to a process for the preparation of a compound of formula (I) comprising following step:
a) coupling reaction between compound of formula (II),

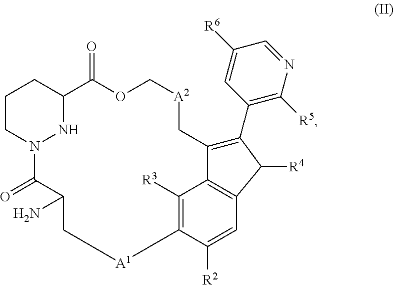

and acid (III),

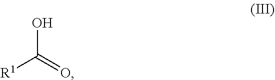

in the presence of a coupling reagent and a base to form the compound of formula (I);
wherein
in step a) the coupling reagent can be, for example, T₃P, HATU, PyBOP or EDCI/HOBt; the base can be, for example, TEA, DIEPA or DMAP.

A compound of formula (I) or (Ia) when manufactured according to the above process is also an object of the invention.

### EXAMPLES

The invention will be more fully understood by reference to the following examples. They should not, however, be construed as limiting the scope of the invention.

### Abbreviations

The invention will be more fully understood by reference to the following examples. They should not, however, be construed as limiting the scope of the invention.

Abbreviations used herein are as follows:
ACN acetonitrile
aq. Aqueous
Boc-N-Me-Val-OH N-(tert-Butoxycarbonyl)-N-methyl-L-valine
(Boc)₂O Di-tert-butyldicarbonate
(R)-binap (R)-(+)-2,2′-Bis(diphenylphosphino)-1,1′-binaphthyl
CDCl₃: deuterated chloroform
CD₃OD: deuterated methanol
COMU (1-Cyano-2-ethoxy-2-oxoethylidenaminooxy)dimethylamino-morpholino-carbenium hexafluorophosphate
DIEPA: N, N-diethylpropylamine
DIBAL-H Diisobutylaluminium hydride
DMAP: 4-Dimethylaminopyridine
DMF: dimethyl formamide
DMP 1,1,1-Tris(acetyloxy)-1,1-dihydro-1,2-benziodoxol-3-(1H)-one
DMSO: dimethyl sulfoxide
EDCI: N-Ethyl-N′-(3-dimethylaminopropyl)carbodiimide hydrochloride
EtOAc or EA: ethyl acetate
FRET fluorescence resonance energy transfer
HATU: (1-[Bis(dimethylamino)methylene]-1H-1,2,3-triazolo[4,5-b]pyridinium 3-oxid hexafluorophosphate)
hr(s): hour(s)
HPLC: high performance liquid chromatography
HOBt: N-hydroxybenzotriazole
H-VAL-OTBU HCl (S)-tert-Butyl 2-amino-3-methylbutanoate hydrochloride
[Ir(OMe)(COD)]₂ (1,5-Cyclooctadiene)(methoxy)iridium(I) dimer
LDA Lithium diisopropylamide
MS: (ESI): mass spectroscopy (electron spray ionization)
min(s) minute(s)
MTBE Methyl tert-butyl ether
NMM N-Methylmorpholine
NMR: nuclear magnetic resonance
NMO 4-Methylmorpholine N-oxide
obsd. Observed
Pd(dppf)Cl₂ [1,1′-Bis(diphenylphosphino)ferrocene]dichloropalladium(II)
Pd(dtbpf)Cl₂ [1,1′-Bis(di-tert-butylphosphino)ferrocene]dichloropalladium(II)
preparative high performance liquid chromatography prep-HPLC
PyBOP: benzotriazol-1-yloxytripyrrolidinophosphonium hexafluorophosphate
RT or rt: room temperature
sat. saturated
SFC supercritical fluid chromatography
TEA: triethylamine
TFA: trifluoroacetic acid
THF: tetrahydrofuran
TEA: trimethylamine
TMEDA Tetramethylethylenediamine
TMSCF₃ Trifluoromethyltrimethylsilane
T₃P: propylphosphonic anhydride

### General Experimental Conditions

Intermediates and final compounds were purified by flash chromatography using one of the following instruments: i) Biotage SPI system and the Quad 12/25 Cartridge module, ii) ISCO combi-flash chromatography instrument. Silica gel brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃·H₂O in MeOH), back pressure 100 bar, detection UV@254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃·H₂O in H₂O; B: acetonitrile;
Basic condition II: A: 0.025% NH₃·H₂O in H₂O; B: acetonitrile;
Neutral condition: A: H₂O; B: acetonitrile.

Mass spectra (MS): generally only ions which indicate the parent mass are reported, and unless otherwise stated the mass ion quoted is the positive mass ion (MH)⁺.

NMR Spectra were obtained using Bruker Avance 400 MHz or 500 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### PREPARATIVE EXAMPLES

### Preparation of Intermediate

### Intermediate A

### 1-[6-[(1S)-1-methoxyethyl]-5-(4,4,5,5-tetramethyl-1,3,2-dioxaborolan-2-yl)-3-pyridyl]-4-methyl-piperazine

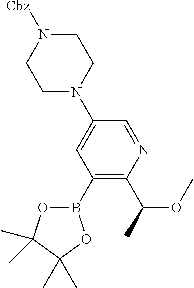

The title intermediate A was prepared according to the following scheme:

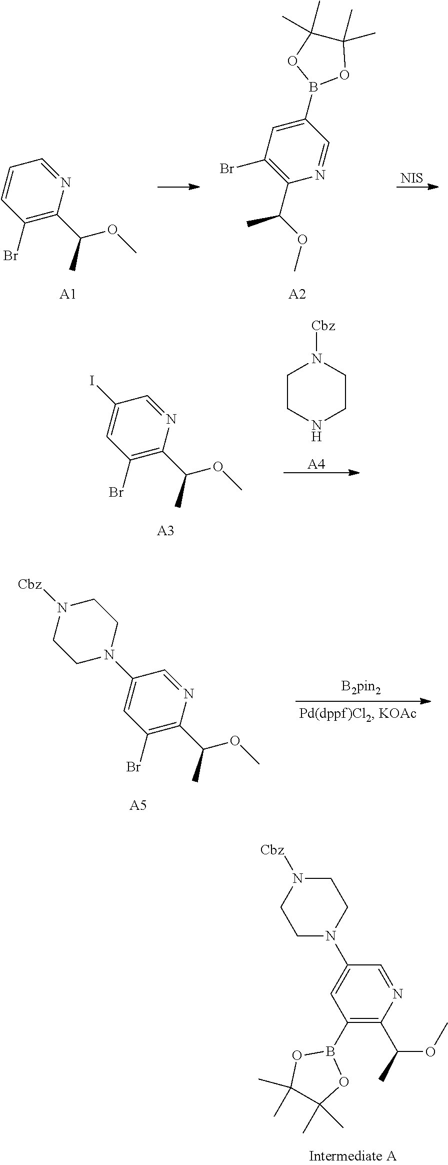

### Step 1: Preparation of 3-bromo-2-[(1S)-1-methoxyethyl]-5-(4,4,5,5-tetramethyl-1,3,2-dioxaborolan-2-yl)pyridine (Compound A2)

To a solution of 3-bromo-2-[(1S)-1-methoxyethyl]pyridine (compound A1, 2.0 g, 9.26 mmol) and bis(pinacolato)diboron (3.5 g, 13.9 mmol) in THF (30 mL) were added 4,4′-di-tert-butyl-2,2′-bipyridin (372.7 mg, 1.39 mmol) and [Ir(OMe)(COD)]₂ (306.3 mg, 0.460 mmol). The mixture was stirred at 75° C. for 16 hours under N₂ protection. The mixture was filtrated and the filtrate was concentrated in vacuo. The residue was purified by silica gel chromatography (EA/PE: 0-20%) to afford 3-bromo-2-[(1S)-1-methoxyethyl]-5-(4,4,5,5-tetramethyl-1,3,2-dioxaborolan-2-yl)pyridine (compound A2, 2.4 g) as yellow oil. ¹H NMR (400 MHZ, CDCl3) δ ppm 8.91 (d, J=1.4 Hz, 1H), 8.21 (d, J=1.4 Hz, 1H), 4.95 (q, J=6.5 Hz, 1H), 3.30 (s, 3H), 1.49 (d, J=6.5 Hz, 3H), 1.35 (s, 12H).

### Step 2: Preparation of 3-bromo-5-iodo-2-[(1S)-1-methoxyethyl]pyridine (Compound A3)

To a solution of 3-bromo-2-[(1S)-1-methoxyethyl]-5-(4,4,5,5-tetramethyl-1,3,2-dioxaborolan-2-yl)pyridine (compound A2, 2.5 g, 7.3 mmol) in ACN (40 mL) was added N-iodosuccinimide (4.1 g, 18.27 mmol). The mixture was stirred at 90° C. for 40 hrs under N₂ protection. The reaction was quenched with saturated solution of Na₂SO₃ (40 mL) and the reaction mixture was extracted with EtOAc (30 mL, twice). The combined organic layer was washed with brine (50 mL), filtered and the filtrate was concentrated under vacuum. The residue was purified by silica gel chromatography (EA/PE: 0-20%) to afford 3-bromo-5-iodo-2-[(1S)-1-methoxyethyl]pyridine (compound A3, 660 mg) as yellow oil. MS calc'd 342 (MH⁺), measured 341.8 (MH⁺).

### Step 3: Preparation of benzyl 4-[5-bromo-6-[(1S)-1-methoxyethyl]-3-pyridyl]piperazine-1-carboxylate (Compound A5)

To a solution of 3-bromo-5-iodo-2-[(1S)-1-methoxyethyl]pyridine (compound A3, 660 mg, 1.9 mmol) and 1-Cbz-piperazine (compound A4, 425.1 mg, 1.9 mmol) in toluene (10 mL) were added cesium carbonate (1.6 g, 4.83 mmol), (R)-BINAP (60.1 mg, 0.1 mmol) and palladium (II) acetate (43.3 mg, 0.19 mmol). The mixture was stirred at 100° C. for 12 hours under N₂ protection. The mixture was filtered and the filtrate was concentrated under vacuum. The residue was purified by silica gel chromatography (EA/PE: 0-50%) to afford benzyl 4-[5-bromo-6-[(1S)-1-methoxyethyl]-3-pyridyl]piperazine-1-carboxylate (compound A5, 740 mg) as a yellow solid. MS calc'd 434.1 (MH⁺), measured 434.1 (MH⁺).

### Step 4: Preparation of 1-[6-[(1S)-1-methoxyethyl]-5-(4,4,5,5-tetramethyl-1,3,2-dioxaborolan-2-yl)-3-pyridyl]-4-methyl-piperazine (Intermediate A)

To a solution of benzyl 4-[5-bromo-6-[(1S)-1-methoxyethyl]-3-pyridyl]piperazine-1-carboxylate (compound A5, 740 mg, 1.7 mmol) and bis(pinacolato)diboron (519.2 mg, 2.04 mmol) in toluene (12 mL) were added KOAc (418.0 mg, 4.26 mmol) and Pd(dppf)Cl₂ (124.7 mg, 0.170 mmol). The reaction mixture was stirred at 90° C. for 12 hrs under N₂ protection. The mixture was filtered and the filtrate was concentrated in vacuo. The residue was purified by silica gel column to afford 1-[6-[(1S)-1-methoxyethyl]-5-(4,4,5,5-tetramethyl-1,3,2-dioxaborolan-2-yl)-3-pyridyl]-4-methyl-piperazine (Intermediate A, 470 mg) as a brown solid. MS calc'd 482.3 (MH⁺), measured 482.2 (MH⁺).

### Intermediate B

### Methyl (3S)-1-[(2S)-3-(4-bromothiazol-2-yl)-2-(tert-butoxycarbonylamino)-propanoyl]hexahydropyridazine-3-carboxylate

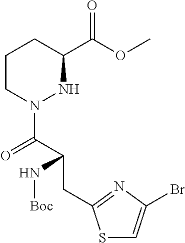 [SMILES: COC(=O)[C@@H]1CCCN(C(=O)[C@H](Cc2nc(Br)cs2)NC(=O)OC(C)(C)C)N1]

The intermediate B was prepared according to the following scheme:

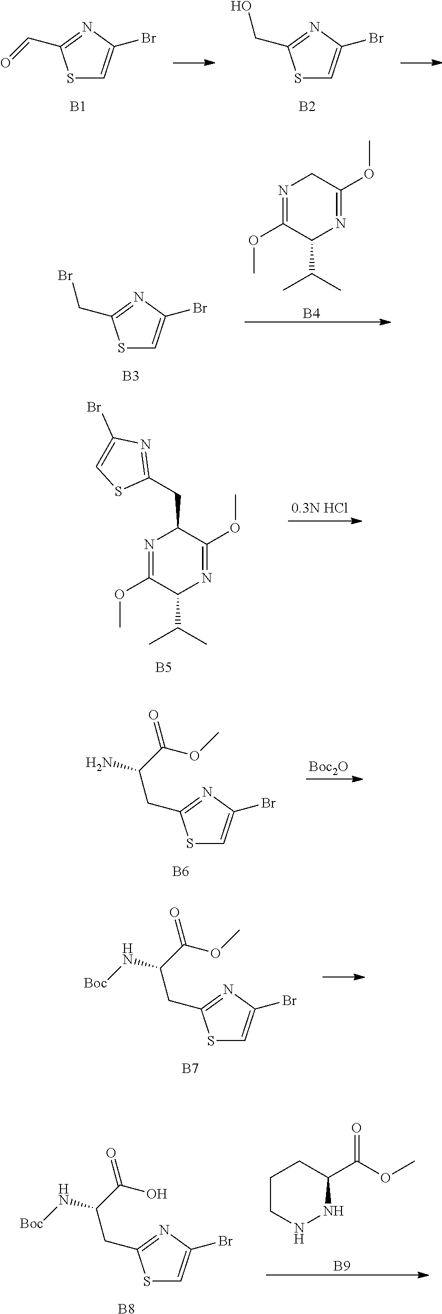

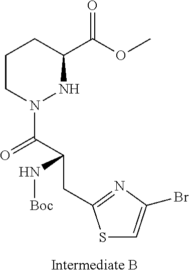 [SMILES: COC(=O)[C@@H]1CCCN(C(=O)[C@H](Cc2nc(Br)cs2)NC(=O)OC(C)(C)C)N1]

### Step 1: Preparation of (4-bromothiazol-2-yl) methanol (Compound B2)

To a solution of 4-bromothiazole-2-carboxaldehyde (compound B1, 6.0 g, 31.25 mmol) in methanol (70 mL) was added sodium borohydride (1.7 g, 46.87 mmol) at 0° C. The mixture was stirred at 25° C. for 1 hour. The reaction was quenched with water (300 mL) at 0° C. and the reaction mixture was extracted by ethyl acetate (200 mL, three times). The combined organic phase was washed with brine (150 mL, twice), dried over anhydrous sodium sulfate, filtered and the filtrate was concentrated under vacuum to afford (4-bromothiazol-2-yl) methanol (compound B2, 6 g) as colorless oil.

### Step 2: Preparation of 4-bromo-2-(bromomethyl)thiazole (Compound B3)

To a solution of (4-bromothiazol-2-yl) methanol (compound B2, 6.0 g, 30.92 mmol) in DCM (80 mL) was added CBr₄ (15.4 g, 46.38 mmol) and triphenylphosphine (12.1 g, 46.38 mmol) at 0° C. After being stirred at 25° C. for 1 hour, the mixture was filtered and the filtrate was concentrated under vacuum. The residue was purified by silica gel column, eluted with ethyl acetate in petroleum ether (0˜10%) to afford (4-bromothiazol-2-yl) methanol (compound B3, 6.0 g) as yellow oil. MS calc'd 255.9 (MH⁺), measured 255.9 (MH⁺).

### Step 3: Preparation of 4-bromo-2-[[(2S,5R)-5-isopropyl-3,6-dimethoxy-2,5-dihydropyrazin-2-yl]methyl]thiazole (Compound B5)

To a mixture of (R)-2,5-dihydro-3,6-dimethoxy-2-isopropylpyrazine (compound B4, 4.3 g, 23.45 mmol) in THF (60 mL) was added n-butyllithium (10 mL, 25.22 mmol, 2.5 M) at −78° C. slowly. After addition, the mixture was stirred for 0.5 hour at −78° C. 4-bromo-2-(bromomethyl)thiazole (compound B3, 5.4 g, 21.02 mmol) was added into above mixture at −78° C. which was stirred for another 1 hour. The reaction was quenched with saturated solution of NH₄Cl (100 mL) and the reaction mixture was extracted with EtOAc (100 mL, twice). The combined organic layer was washed with brine (150 mL), dried over anhydrous sodium sulfate, filtered and the filtrate was concentrated under vacuum. The residue was purified by reversed-phase chromatography to afford 4-bromo-2-[[(2S,5R)-5-isopropyl-3,6-dimethoxy-2,5-dihydropyrazin-2-yl]methyl]thiazole (compound B5, 3.6 g) as yellow oil. MS calc'd 360 (MH⁺), measured 359.9 (MH⁺).

### Step 4: Preparation of methyl (2S)-2-amino-3-(4-bromothiazol-2-yl)propanoate (Compound B6)

To a solution of 4-bromo-2-[[(2S,5R)-5-isopropyl-3,6-dimethoxy-2,5-dihydropyrazin-2-yl]methyl]thiazole (compound B5, 3.6 g, 10 mmol) in ACN (20 mL) was added hydrochloric acid (66.6 mL, 0.3 M). The mixture was stirred at 25° C. for 2 hours. The mixture was basified by saturated solution of NaHCO₃ until pH=8. The mixture was extracted with EtOAc (80 mL, six times). The combined organic layer was dried over anhydrous sodium sulfate, filtered and the filtrate was concentrated under vacuum to afford methyl (2S)-2-amino-3-(4-bromothiazol-2-yl)propanoate (compound B6, 3.1 g) as yellow oil. MS calc'd 264.9 (MH⁺), measured 264.9 (MH⁺).

### Step 5: Preparation of methyl (2S)-3-(4-bromothiazol-2-yl)-2-(tert-butoxycarbonylamino)propanoate (Compound B7)

To a solution of methyl (2S)-2-amino-3-(4-bromothiazol-2-yl)propanoate (compound B6, 3.1 g, 11.69 mmol) in DCM (40 mL) were added triethylamine (2.9 g, 29.23 mmol) and (Boc)₂O (3.8 g, 17.54 mmol). After being stirred at 30° C. for 12 hours, the mixture was concentrated under vacuum. The residue was purified by silica gel column, eluted with ethyl acetate in petroleum ether (0˜30%) to afford methyl (2S)-3-(4-bromothiazol-2-yl)-2-(tert-butoxycarbonylamino)propanoate (compound B7, 3.2 g) as yellow oil. MS calc'd 387 (MNa⁺), measured 386.9 (MNa⁺).

### Step 6: Preparation of (2S)-3-(4-bromothiazol-2-yl)-2-(tert-butoxycarbonylamino)-propanoic acid (Compound B8)

To a solution of methyl (2S)-3-(4-bromothiazol-2-yl)-2-(tert-butoxycarbonylamino)propanoate (compound B7, 3.2 g, 8.76 mmol) in THF (30 mL), methanol (2 mL) and water (10 mL) was added lithium hydroxide (0.4 mL, 43.81 mmol). After being stirred at 25° C. for 1 hour, the reaction mixture was acidified by 1 M solution of HCl until pH=5. The mixture was extracted with EtOAc (40 mL, twice). The combined organic layer was washed with brine (100 mL), dried over anhydrous sodium sulfate, filtered and the filtrate was concentrated under vacuum to afford (2S)-3-(4-bromothiazol-2-yl)-2-(tert-butoxycarbonylamino)propanoic acid (compound B8, 3.1 g) as yellow oil. MS calc'd 373 (MNa⁺), measured 372.9 (MNa⁺).

### Step 7: Preparation of methyl (3S)-1-[(2S)-3-(4-bromothiazol-2-yl)-2-(tert-butoxycarbonylamino)propanoyl]hexahydropyridazine-3-carboxylate (Intermediate B)

To a solution of (2S)-3-(4-bromothiazol-2-yl)-2-(tert-butoxycarbonylamino)propanoic acid (compound B8, 3.1 g, 8.83 mmol) in DCM (50 mL) was added methyl (3S)-hexahydropyridazine-3-carboxylate; hydrochloride (compound B9, 2.4 g, 13.24 mmol), EDCI (3.4 g, 17.65 mmol), 1-Hydroxybenzotriazole (238.5 mg, 1.77 mmol) and NMM (9.92 mL, 88.26 mmol) at 0° C. After being stirred at 25° C. for 1 hour, the reaction mixture was diluted with water (60 mL) and extracted with EtOAc (60 mL, three times). The combined organic layer was washed with brine (100 mL), dried over anhydrous sodium sulfate, filtered and the filtrate was concentrated under vacuum. The residue was purified by silica gel column and eluted with ethyl acetate in petroleum ether (10˜30%) to afford methyl (3S)-1-[(2S)-3-(4-bromothiazol-2-yl)-2-(tert-butoxycarbonylamino)propanoyl]hexahydropyridazine-3-carboxylate (intermediate B, 2.4 g). MS calc'd 477 (MH⁺), measured 476.9 (MH⁺).

### Intermediate C

### (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26]octacosa-}1(25),2,5(28),19,22(26),23-hexaene-8,14-dione

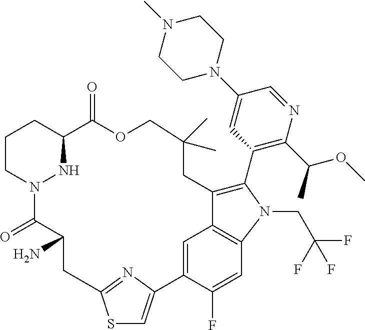

The title intermediate C was prepared according to the following scheme:

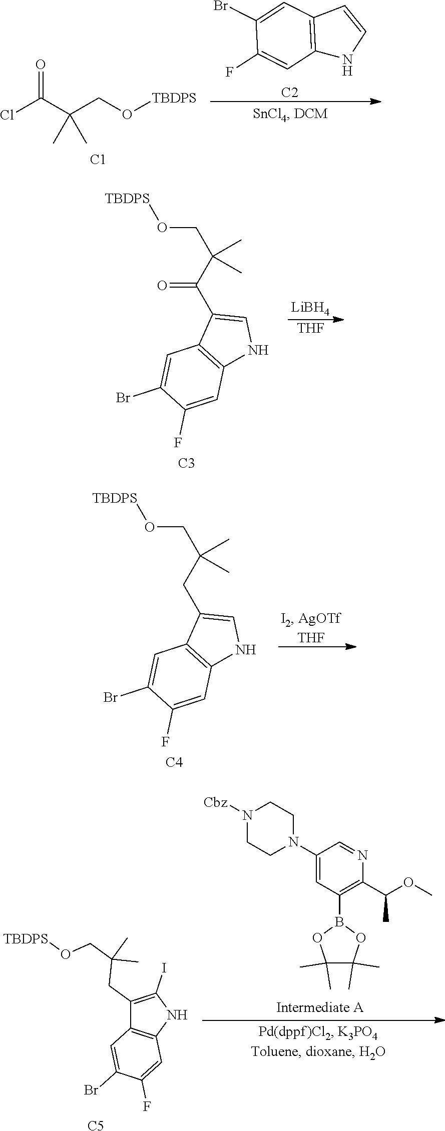

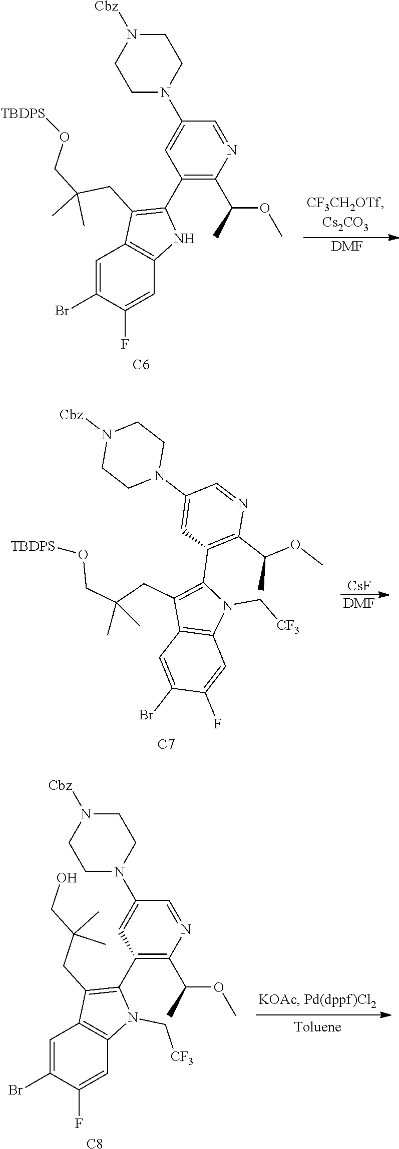

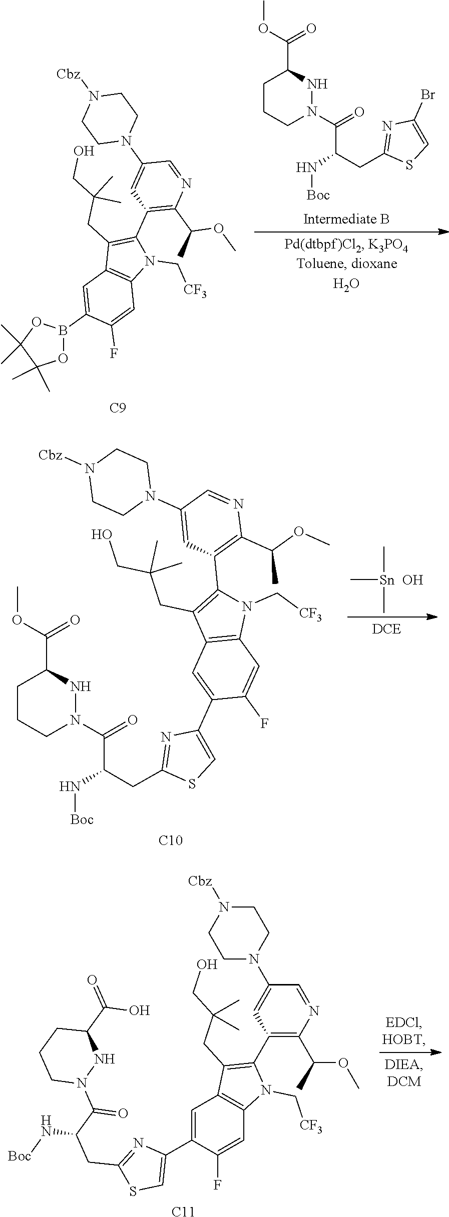

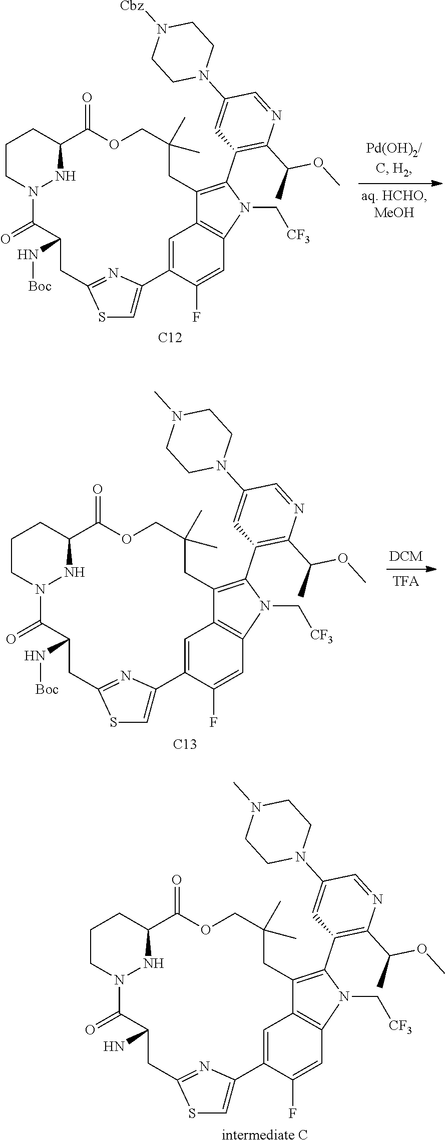

### Step 1: Preparation of 1-(5-bromo-6-fluoro-1H-indol-3-yl)-3-((tert-butyldiphenylsilyl)oxy)-2,2-dimethylpropan-1-one (compound C3)

To a mixture of 3-((tert-butyldiphenylsilyl)oxy)-2,2-dimethylpropanoyl chloride (compound C1, 35.0 g, 116.8 mmol) in DCM (400 mL) at 0° C. was added a solution of SnCl₄ (97.2 mL, 121.5 mmol) slowly. After being stirred at −40° C. for 0.5 hour, 5-bromo-6-fluoro-1H-indole (compound C2, 25.0 g, 116.8 mmol) in DCM (200 mL) was added dropwise to the mixture which was stirred at −40° C. for 15 min. After the reaction was completed, it was quenched with sat. NaHCO₃ aq. (800 mL), and the reaction mixture was extracted with EtOAc (900 mL, twice). The combined organic layer was washed with brine (700 mL), dried over Na₂SO₄, filtered and concentrated in vacuo. The residue was triturated with the solution (100 mL, Petroleum ether: Ethyl acetate=8:1) and filtered. The filter cake was dried in vacuo to afford 1-(5-bromo-6-fluoro-1H-indol-3-yl)-3-((tertbutyldiphenylsilyl)oxy)-2,2-dimethylpropan-1-one (compound C3, 50.0 g) as a yellow solid. MS calc'd 552.1 (MH⁺), measured 552.1 (MH⁺).

### Step 2: Preparation of [3-(5-bromo-6-fluoro-1H-indol-3-yl)-2,2-dimethyl-propoxy]-tert-butyl-diphenyl-silane (compound C4)

To a mixture of 1-(5-bromo-6-fluoro-1H-indol-3-yl)-3-((tertbutyldiphenylsilyl)oxy)-2,2-dimethylpropan-1-one (compound C3, 50.0 g, 90.49 mmol) in THF (600 mL) was added LiBH₄ (48.4 mL, 193.49 mmol, 4 M in THF) dropwise at 0° C. The mixture was stirred at 70° C. for 24 hrs under nitrogen atmosphere. After the reaction was completed, it was quenched by addition of water (600 mL) at 0° C. slowly and the reaction mixture was extracted with EtOAc (600 mL, twice). The combined organic layer was washed with brine (600 mL), dried over Na₂SO₄, filtered and concentrated in vacuo. The residue was purified by silica column chromatography (EtOAc in PE=20%˜33%) to afford [3-(5-bromo-6-fluoro-1H-indol-3-yl)-2,2-dimethyl-propoxy]-tert-butyl-diphenyl-silane (compound C4, 46.0 g) as a white solid. MS calc'd 538.1 (MH⁺), measured 538.2 (MH⁺).

### Step 3: Preparation of [3-(5-bromo-6-fluoro-2-iodo-1H-indol-3-yl)-2,2-dimethyl-propoxy]-tert-butyl-diphenyl-silane (compound C5)

To a mixture of [3-(5-bromo-6-fluoro-1H-indol-3-yl)-2,2-dimethyl-propoxy]-tert-butyl-diphenyl-silane (compound C4, 35.4 g, 65.73 mmol) and iodine (18.4 g, 72.3 mmol) in THF (400 mL) was added silver trifluoromethanesulfonate (20.3 g, 78.88 mmol) at 0° C. The mixture was stirred at 0° C. for 10 min. After the reaction was completed, it was quenched by sat. Na₂SO₃ aq. (400 mL) and EtOAc (400 mL) and the reaction mixture was filtered. The organic layer was washed with brine (100 mL), dried over Na₂SO₄, filtered and concentrated in vacuo. The residue was purified by silica column chromatography (EtOAc in PE=0%˜2.5%) to afford [3-(5-bromo-6-fluoro-2-iodo-1H-indol-3-yl)-2,2-dimethyl-propoxy]-tert-butyl-diphenyl-silane (compound C5, 43.0 g) as a yellow solid. MS calc'd 664.0 (MH⁺), measured 664.1 (MH⁺).

### Step 4: Preparation of benzyl 4-[5-[5-bromo-3-[3-[tert-butyl(diphenyl)silyl]oxy-2,2-dimethyl-propyl]-6-fluoro-1H-indol-2-yl]-6-[(1S)-1-methoxyethyl]-3-pyridyl]piperazine-1-carboxylate (Compound C6)

To a mixture of [3-(5-bromo-6-fluoro-2-iodo-1H-indol-3-yl)-2,2-dimethyl-propoxy]-tert-butyl-diphenyl-silane (compound C5, 16.7 g, 25.13 mmol) and benzyl 4-[6-[(1S)-1-methoxyethyl]-5-(4,4,5,5-tetramethyl-1,3,2-dioxaborolan-2-yl)-3-pyridyl]piperazine-1-carboxylate (Intermediate A, 16.7 g, 34.69 mmol) in a mixed solution of 1,4-dioxane (270 mL)/Toluene (90 mL)/water (90 mL) were added potassium phosphate (15.7 g, 73.92 mmol) and Pd(dppf)Cl₂ (920 mg, 1.26 mmol). The mixture was stirred at 70° C. for 12 hrs under nitrogen atmosphere. After the reaction was completed, the mixture was filtered and concentrated in vacuo. The residue was purified by silica column chromatography (EtOAc in PE=20%˜50%) to afford 4-[5-[5-bromo-3-[3-[tert-butyl(diphenyl)silyl]oxy-2,2-dimethyl-propyl]-6-fluoro-1H-indol-2-yl]-6-[(1S)-1-methoxyethyl]-3-pyridyl]piperazine-1-carboxylate (compound C6, 19.5 g) as a white solid. MS calc'd 891.3 (MH′), measured 891.3 (MH′).

### Step 5: Preparation of benzyl 4-[(5M)-5-[5-bromo-3-[3-[tert-butyl(diphenyl)silyl]oxy-2,2-dimethyl-propyl]-6-fluoro-1-(2,2,2-trifluoroethyl)indol-2-yl]-6-[(1S)-1-methoxyethyl]-3-pyridyl]piperazine-1-carboxylate (Compound C7)

To a solution of 4-[5-[5-bromo-3-[3-[tert-butyl(diphenyl)silyl]oxy-2,2-dimethyl-propyl]-6-fluoro-1H-indol-2-yl]-6-[(1S)-1-methoxyethyl]-3-pyridyl]piperazine-1-carboxylate (compound C6, 14.5 g, 16.26 mmol) and Cs₂CO₃ (15.9 g, 48.77 mmol) in DMF (200 mL) was added 2,2,2-trifluoroethyl trifluoromethanesulfonate (37.7 g, 162.56 mmol) dropwise at 0° C., and the mixture was stirred at 20° C. for 12 hrs. After the reaction was completed, EtOAc (70 mL) and water (100 mL) were added and the layers were separated. The aqueous phase was extracted with EtOAc (70 mL, twice). Combined organic layer was washed with brine (100 mL, four times), dried over Na₂SO₄, filtered, and concentrated under vacuum to give a residue. The residue was purified by silica column chromatography to afford benzyl 4-[(5M)-5-[5-bromo-3-[3-[tert-butyl(diphenyl)silyl]oxy-2,2-dimethyl-propyl]-6-fluoro-1-(2,2,2-trifluoroethyl)indol-2-yl]-6-[(1S)-1-methoxyethyl]-3-pyridyl]piperazine-1-carboxylate (compound C7, 8.0 g, PEAK 1, faster eluted) as yellow oil. MS calc'd 973.3 (MH⁺), measured 973.2 (MH⁺).

### Step 6: Preparation of benzyl 4-[(5M)-5-[5-bromo-6-fluoro-3-(3-hydroxy-2,2-dimethyl-propyl)-1-(2,2,2-trifluoroethyl)indol-2-yl]-6-[(1S)-1-methoxyethyl]-3-pyridyl]piperazine-1-carboxylate (Compound C8)

To a solution of benzyl 4-[(5M)-5-[5-bromo-3-[3-[tert-butyl(diphenyl)silyl]oxy-2,2-dimethyl-propyl]-6-fluoro-1-(2,2,2-trifluoroethyl)indol-2-yl]-6-[(1S)-1-methoxyethyl]-3-pyridyl]piperazine-1-carboxylate (compound C7, 10.5 g, 10.78 mmol) in DMF (130 mL) was added cesium fluoride (8.2 g, 53.9 mmol) and the mixture was stirred at 60° C. for 24 hrs. After the reaction was completed. EtOAc (100 mL) and water (100 mL) were added and the layers were separated. The aqueous phase was extracted with EtOAc (100 mL, twice). The combined organic layer was washed with brine (80 mL, three times), dried over Na₂SO₄, filtered, and concentrated under vacuum to give a residue. The residue was purified by silica column chromatography (EtOAc in PE=25%˜66%) to afford benzyl 4-[(5M)-5-[5-bromo-6-fluoro-3-(3-hydroxy-2,2-dimethyl-propyl)-1-(2,2,2-trifluoroethyl)indol-2-yl]-6-[(1S)-1-methoxyethyl]-3-pyridyl]piperazine-1-carboxylate (compound C8, 6.5 g) as a yellow solid. MS calc'd 735.2 (MH⁺), measured 735.1 (MH⁺).

### Step 7: Preparation of benzyl 4-[(5M)-5-[6-fluoro-3-(3-hydroxy-2,2-dimethyl-propyl)-5-(4,4,5,5-tetramethyl-1,3,2-dioxaborolan-2-yl)-1-(2,2,2-trifluoroethyl)indol-2-yl]-6-[(1S)-1-methoxyethyl]-3-pyridyl]piperazine-1-carboxylate (compound C9)

To a solution of benzyl 4-[(5M)-5-[5-bromo-6-fluoro-3-(3-hydroxy-2,2-dimethyl-propyl)-1-(2,2,2-trifluoroethyl)indol-2-yl]-6-[(1S)-1-methoxyethyl]-3-pyridyl]piperazine-1-carboxylate (compound C8, 5.4 g), bis(pinacolato)diboron (2.8 g, 11.01 mmol) and potassium acetate (1.2 mL, 18.35 mmol) in toluene (70 mL) was added Pd(dppf)Cl₂ (537.1 mg, 0.73 mmol). The mixture was degassed and purged with nitrogen atmosphere for three times and the mixture was stirred at 90° C. for 12 hrs. After the reaction was completed, the mixture was cooled to room temperature. The reaction mixture was filtered and the filtrate was concentrated in vacuo to give a residue. The residue was purified by silica column chromatography (EtOAc in PE=25%˜66%) to afford benzyl 4-[(5M)-5-[6-fluoro-3-(3-hydroxy-2,2-dimethyl-propyl)-5-(4,4,5,5-tetramethyl-1,3,2-dioxaborolan-2-yl)-1-(2,2,2-trifluoroethyl)indol-2-yl]-6-[(1S)-1-methoxyethyl]-3-pyridyl]piperazine-1-carboxylate (compound C9, 5.2 g) as yellow oil. MS calc'd 783.3 (MH⁺), measured 783.3 (MH⁺).

### Step 8: Preparation of methyl (3S)-1-[(2S)-3-[4-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]thiazol-2-yl]-2-(tert-butoxycarbonylamino)-propanoyl]hexahydropyridazine-3-carboxylate (compound C10)

To a mixture of methyl (3S)-1-[(2S)-3-(4-bromothiazol-2-yl)-2-(tert-butoxycarbonylamino)propanoyl]hexahydropyridazine-3-carboxylate (intermediate B, 2.7 g, 5.69 mmol), benzyl 4-[(5M)-5-[6-fluoro-3-(3-hydroxy-2,2-dimethyl-propyl)-5-(4,4,5,5-tetramethyl-1,3,2-dioxaborolan-2-yl)-1-(2,2,2-trifluoroethyl)indol-2-yl]-6-[(1S)-1-methoxyethyl]-3-pyridyl]piperazine-1-carboxylate (compound C9, 4.9 g, 6.32 mmol) in toluene (60 mL)/1,4-dioxane (20 mL)/water (20 mL) were added K₃PO₄ (3.4 g, 15.81 mmol) and Pd(dtbpf)Cl₂ (412.2 mg, 0.63 mmol) under nitrogen atmosphere. The mixture was stirred at 70° C. for 12 hrs. After the reaction was completed, the mixture was concentrated in vacuo to give a residue. The residue was purified by silica column (EtOAc in PE=10%˜75%) to afford methyl (3S)-1-[(2S)-3-[4-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]thiazol-2-yl]-2-(tert-butoxycarbonylamino)-propanoyl]hexahydropyridazine-3-carboxylate (compound C10, 3.6 g) as a brown solid. MS calc'd 1053.4 (MH⁺), measured 1053.3 (MH⁺).

### Step 9: Preparation of (3S)-1-[(2S)-3-[4-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]thiazol-2-yl]-2-(tert-butoxycarbonylamino)propanoyl]hexahydropyridazine-3-carboxylic acid (Compound C11)

To a solution of methyl (3S)-1-[(2S)-3-[4-[(2M)-2-[5-(4-benzyloxy carbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]thiazol-2-yl]-2-(tert-butoxycarbonylamino)-propanoyl]-hexahydropyridazine-3-carboxylate (compound C10, 3.6 g, 3.42 mmol) in DCE (50 mL) was added trimethylstannanol (2.4 g, 13.67 mmol) and the mixture was stirred at 60° C. for 12 hrs. After the reaction was completed. EtOAc (80 mL) and water (60 mL) were added and the layers were separated. The aqueous phase was extracted with EtOAc (80 mL, twice). The combined organic layer was washed with brine (100 mL), dried over Na₂SO₄, filtered, and concentrated under vacuum to give (3S)-1-[(2S)-3-[4-[(2M)-2-[5-(4-benzyloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]thiazol-2-yl]-2-(tert-butoxycarbonylamino)propanoyl]hexahydropyridazine-3-carboxylic acid (compound C11, 4.3 g) as a brown solid. MS calc'd 1039.4 (MH⁺), measured 1039.2 (MH⁺).

### Step 10: Preparation of benzyl 4-[5-[(7S,13S)-7-(tert-butoxycarbonylamino)-24-fluoro-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26]octacosa-}1(25),2,5(28),19,22(26),23-hexaen-(20M)-20-yl]-6-[(1S)-1-methoxyethyl]-3-pyridyl]piperazine-1-carboxylate (Compound C12)

To a mixture of (3S)-1-[(2S)-3-[4-[(2M)-2-[5-(4-benzloxycarbonylpiperazin-1-yl)-2-[(1S)-1-methoxyethyl]-3-pyridyl]-6-fluoro-3-(3-hydroxy-2,2-dimethyl-propyl)-1-(2,2,2-trifluoroethyl)indol-5-yl]thiazol-2-yl]-2-(tert-butoxycarbonylamino)propanoyl]hexahydropyridazine-3-carboxylic acid (compound C11, 4.3 g, 4.14 mmol) in DCM (430 mL) was added DIEA (14.4 mL, 82.76 mmol), EDCI (11.9 g, 62.07 mmol) and 1-hydroxybenzotriazole (1.4 g, 10.35 mmol) at (° C.) The mixture was stirred at 15° C. for 12 hrs. After the reaction was completed, the mixture was concentrated in vacuo, then diluted with water (80) mL), extracted with EtOAc (80 mL, twice). The combined organic layer was washed with brine (80) mL), dried over Na₂SO₄, filtered and concentrated in vacuo. The residue was purified by silica column chromatography (EtOAc in PE=25%˜66%) to afford benzyl 4-[5-[(7S,13S)-7-(tert-butoxycarbonylamino)-24-fluoro-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-(20M)-20-yl]-6-[(1S)-1-methoxyethyl]-3-pyridyl]piperazine-1-carboxylate (compound C12, 3.1 g) as yellow gum. MS calc'd 1021.4 (MH⁺), measured 1021.2 (MH⁺).

### Step 11: Preparation of tert-butyl N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (Compound C13)

To a mixture of benzyl 4-[5-[(7S,13S)-7-(tert-butoxycarbonylamino)-24-fluoro-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-(20M)-20)-yl]-6-[(1S)-1-methoxyethyl]-3-pyridyl]piperazine-1-carboxylate (compound C12, 3.1 g, 3.04 mmol) and formaldehyde aqueous (775.0 mg, 9.55 mmol) in methanol (150) mL) was added Pd(OH)₂ on activated carbon (2.79 g, 3.97 mmol). The mixture was degassed and purged with H₂ three times. The mixture was hydrogenated at 30° C. for 18 hrs. After the reaction was completed, the mixture was filtered and the filtrate was concentrated in vacuo to afford tert-butyl N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound C13, 2.6 g) as a brown solid. MS calc'd 901.3 (MH⁺), measured 901.3 (MH⁺).

### Step 12: Preparation of (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate C)

To a mixture of tert-butyl N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound C13, 2.6 g, 2.89 mmol) in DCM (18 mL) was added TFA (14.0 mL, 181.72 mmol). The mixture was stirred at 15° C. for 0.5 h. After the reaction was completed, the mixture was concentrated in vacuo and diluted with sat. NaHCO₃ (30 mL), extracted with EtOAc (30 mL, three times). The combined organic layer was washed with brine (50 mL), dried over Na₂SO₄, filtered and concentrated in vacuo to afford (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate C, 2.0 g) as a yellow solid, which was used directly in the next step. MS calc'd 801.3 (MH⁺), measured 801.2 (MH⁺)

### Intermediate D

### (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

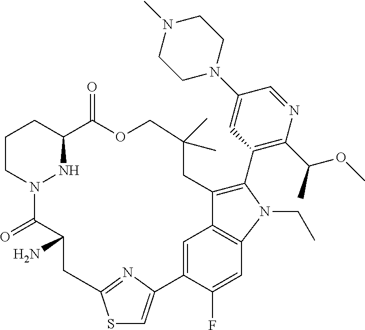

The title compound was prepared in analogy to the preparation of Intermediate C by using iodoethane instead of 2,2,2-trifluoroethyl trifluoromethanesulfonate.

### Intermediate E

### (7S,13S)-7-amino-24-fluoro-(20M)-20-[2-[(1S)-1-methoxyethyl]-5-[4-(2,2,2-trifluoroethyl)piperazin-1-yl]-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

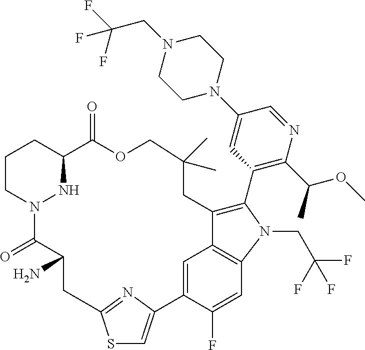

The compound was prepared according to the following scheme:

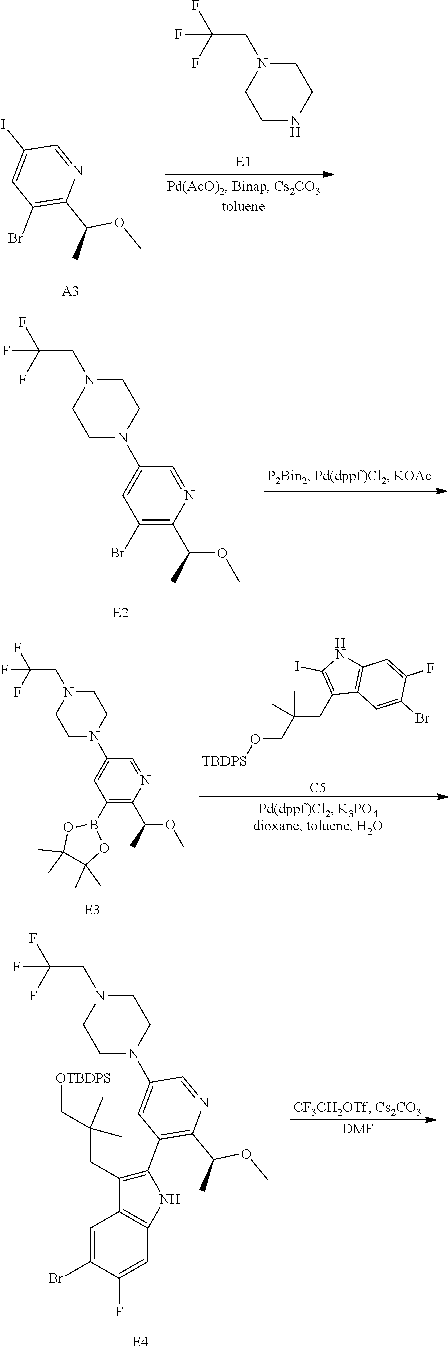

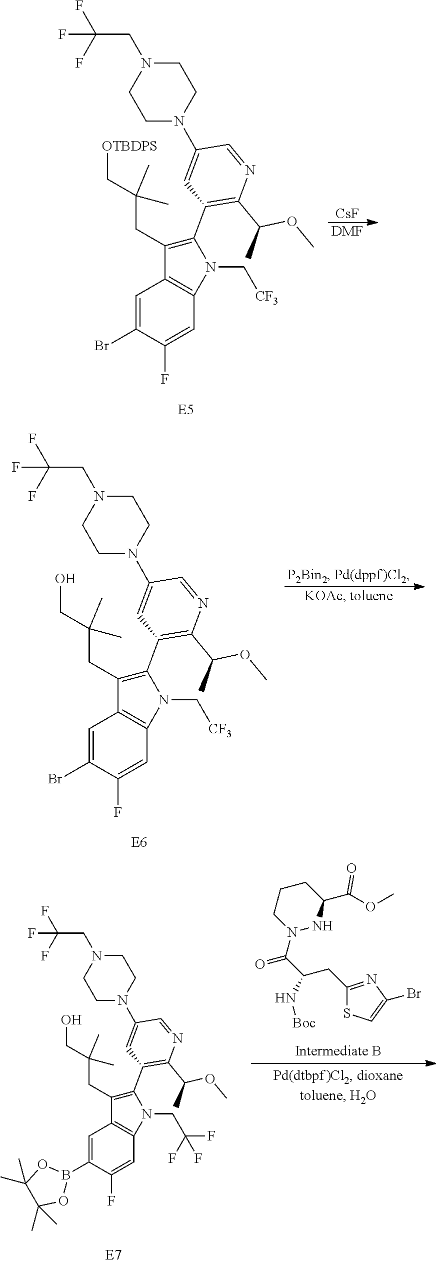

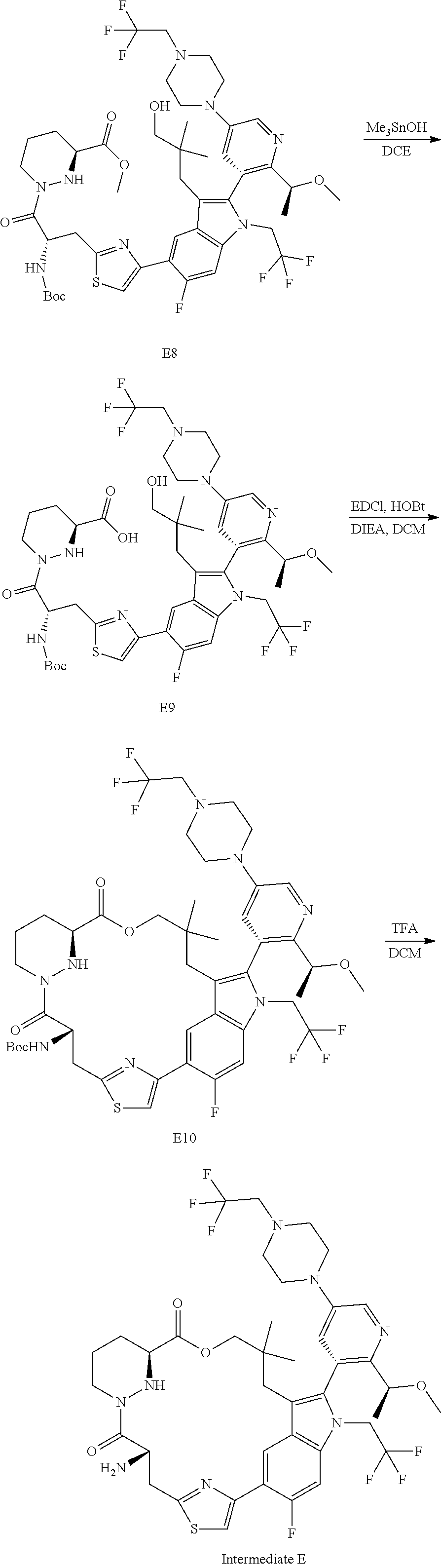

### Step 1: Preparation of 1-[5-bromo-6-[(1S)-1-methoxyethyl]-3-pyridyl]-4-(2,2,2-trifluoroethyl)piperazine (compound E2)

To a mixture of 3-bromo-5-iodo-2-[(1S)-1-methoxyethyl]pyridine (compound A3, 2.03 g, 5.95 mmol) and 1-(2,2,2-trifluoroethyl)piperazine (compound E1, 1.0 g, 5.95 mmol) in toluene (15 mL) were added Cs₂CO₃ (4.85 g, 14.88 mmol), (R)-binap (92.6 mg, 0.15 mmol) and Pd(OAc)₂ (66.8 mg, 0.3 mmol). The reaction mixture was degassed and purged with nitrogen for 3 times and the mixture was stirred at 100° C. for 12 hrs under nitrogen atmosphere. After being cooled to room temperature, the reaction mixture was filtered and the filtrate was concentrated in vacuo to give a residue. The residue was purified by column chromatography to 1-[5-bromo-6-[(1S)-1-methoxyethyl]-3-pyridyl]-4-(2,2,2-trifluoroethyl)piperazine (compound E2, 2.0 g) as yellow oil. MS calc'd 382.2 (MH⁺), measured 382.1 (MH⁺)

### Step 2:1-[6-[(1S)-1-methoxyethyl]-5-(4,4,5,5-tetramethyl-1,3,2-dioxaborolan-2-yl)-3-pyridyl]-4-(2,2,2-trifluoroethyl)piperazine (compound E3)

To a solution of 1-[5-bromo-6-[(1S)-1-methoxyethyl]-3-pyridyl]-4-(2,2,2-trifluoroethyl)piperazine (compound E2, 3.2 g, 8.37 mmol), bis(pinacolato)diboron (3.19 g, 12.56 mmol) and KOAc (2.1 g, 20.93 mmol) in toluene (50 mL) was added Pd(dppf)Cl₂ (306.3 mg, 0.42 mmol). The mixture was degassed and purged with nitrogen for 3 times and the mixture was stirred at 90° C. for 12 hrs under nitrogen atmosphere. After being cooled to the room temperature, the reaction mixture was filtered, the filtrate was concentrated in vacuo to give a residue, which was purified by reversed phase column to afford 1-[6-[(1S)-1-methoxyethyl]-5-(4,4,5,5-tetramethyl-1,3,2-dioxaborolan-2-yl)-3-pyridyl]-4-(2,2,2-trifluoroethyl)piperazine (compound E3, 1.9 g) as a yellow gum. MS calc'd 430.2 (MH⁺), measured 348.4 (M-C₆H₁₀+H⁺).

### Step 3: Preparation of [3-[5-bromo-6-fluoro-2-[2-[(1S)-1-methoxyethyl]-5-[4-(2,2,2-trifluoroethyl)piperazin-1-yl]-3-pyridyl]-1H-indol-3-yl]-2,2-dimethyl-propoxy]-tert-butyl-diphenyl-silane (compound E4)

To a solution of 1-[6-[(1S)-1-methoxyethyl]-5-(4,4,5,5-tetramethyl-1,3,2-dioxaborolan-2-yl)-3-pyridyl]-4-(2,2,2-trifluoroethyl)piperazine (compound E3, 1.9 g, 4.41 mmol), [3-(5-bromo-6-fluoro-2-iodo-1H-indol-3-yl)-2,2-dimethyl-propoxy]-tert-butyl-diphenyl-silane (compound C5, 2.1 g, 3.15 mmol) in 1,4-dioxane (24 mL), water (8 mL) and toluene (8 mL) was added K₃PO₄ (2.1 g, 9.5 mmol) and Pd(dppf)Cl₂ (231 mg, 0.37 mmol). The mixture was degassed by bubbling nitrogen for 2 min, and the reaction mixture was stirred at 70° C. for 12 hrs. After being cooled to 15 room temperature, the reaction mixture was filtered. The filtrate was concentrated in vacuo to give a residue. The residue was purified by column chromatography (EtOAc in PE: 30%-60%) to afford [3-[5-bromo-6-fluoro-2-[2-[(1S)-1-methoxyethyl]-5-[4-(2,2,2-trifluoroethyl)piperazin-1-yl]-3-pyridyl]-1H-indol-3-yl]-2,2-dimethyl-propoxy]-tert-butyl-diphenyl-silane (compound E4, 960.0 mg) as a yellow gum. MS calc'd 839.3 (MH⁺), measured 839.3 (MH⁺)

### Step 4: Preparation of [3-[5-bromo-6-fluoro-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-3-yl]-2,2-dimethyl-propoxy]-tert-butyl-diphenyl-silane (compound E5)

To a solution of [3-[5-bromo-6-fluoro-2-[2-[(1S)-1-methoxyethyl]-5-[4-(2,2,2-trifluoroethyl)piperazin-1-yl]-3-pyridyl]-1H-indol-3-yl]-2,2-dimethyl-propoxy]-ter-butyl-diphenyl-silane (compound E4, 1 g, 1.14 mmol) in DMF (35 mL) was added Cs₂CO₃ (1.1 g, 3.44 mmol) and 2,2,2-trifluoroethyl trifluoromethanesulfonate (2.7 g, 11.63 mmol) at 0° C. After being stirred at 20° C. for 15 hrs, the reaction mixture was poured into water (100) mL), and extracted with EtOAc (50 mL, three times). The combined organic was washed with brine (50 mL, three times), dried over Na₂SO₄, filtered and concentrated under vacuum to give a residue which was purified by column chromatography (EtOAc in PE: 30%-40%) to afford [3-[5-bromo-6-fluoro-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-3-yl]-2,2-dimethyl-propoxy]-tert-butyl-diphenyl-silane (compound E5, 640.0 mg, faster eluted) as a white solid. MS calc'd 921.3 (MH⁺), measured 921.4 (MH⁺).

### Step 5: Preparation of 3-[5-bromo-6-fluoro-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-3-yl]-2,2-dimethyl-propan-1-ol (compound E6)

To a solution of [3-[5-bromo-6-fluoro-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-3-yl]-2,2-dimethyl-propoxy]-tert-butyl-diphenyl-silane (compound E5, 640.0 mg, 0.69 mmol) in DMF (7 mL) was added cesium fluoride (421.8 mg, 2.78 mmol). The mixture was stirred at 60° C. for 16 hrs. After being cooled to room temperature, the reaction mixture was filtered and the filtrate was concentrated in vacuo to give a residue. The residue was purified by column chromatography (EtOAc in PE: 30%-60%) to afford 3-[5-bromo-6-fluoro-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-3-yl]-2,2-dimethyl-propan-1-ol (compound E6, 360.0 mg) as yellow oil. MS calc'd 683.2 (MH⁺), measured 683.1 (MH⁺).

### Step 6: Preparation of 3-[5-bromo-6-fluoro-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-3-yl]-2,2-dimethyl-propan-1-ol (compound E7)

To a solution of 3-[5-bromo-6-fluoro-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-3-yl]-2,2-dimethyl-propan-1-ol (compound E6, 360.0 mg, 0.53 mmol), bis(pinacolato)diboron (200.6 mg, 0.79 mmol) in toluene (6 mL) was added potassium acetate (0.08 mL, 1.32 mmol) and Pd(dppf)Cl₂ (40 mg, 0.1 mmol). The reaction mixture was degassed by bubbling nitrogen for 5 min then stirred at 80° C. for 15 hrs. After being cooled to room temperature, the reaction mixture was filtered and the filtrate was concentrated in vacuo to give a residue. The residue was purified by column chromatography (EtOAc in PE: 30%-50%) to afford 3-[5-bromo-6-fluoro-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-3-yl]-2,2-dimethyl-propan-1-ol (compound E7, 300.0 mg) as yellow gum. MS calc'd 731.4 (MH⁺), measured 731.4 (MH⁺).

### Step 7: Preparation of methyl (3S)-1-[(2S)-2-(tert-butoxycarbonylamino)-3-[4-[6-fluoro-3-(3-hydroxy-2,2-dimethyl-propyl)-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-5-yl]thiazol-2-yl]propanoyl]hexahydropyridazine-3-carboxylate (compound E8)

To a mixture of 3-[5-bromo-6-fluoro-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-3-yl]-2,2-dimethyl-propan-1-ol (compound E7, 0.3 g, 0.41 mmol) and methyl (3S)-1-[(2S)-3-(4-bromothiazol-2-yl)-2-(tert-butoxycarbonylamino)propanoyl]hexahydropyridazine-3-carboxylate (intermediate B, 196.7 mg, 0.41 mmol) in toluene (3 mL), 1,4-dioxane (1 mL) and water (1 mL) were added K₃PO₄ (221.3 mg, 1.04 mmol) and Pd(dtbpf)Cl₂ (27.05 mg, 0.04 mmol). The mixture was stirred at 70° C. for 12 hrs under nitrogen atmosphere. After being cooled to room temperature, the reaction mixture was filtered and the filtrate was concentrated in vacuo to give a residue. The residue was purified by column chromatography (EtOAc in PE: 60%-80%) to afford methyl (3S)-1-[(2S)-2-(tert-butoxycarbonylamino)-3-[4-[6-fluoro-3-(3-hydroxy-2,2-dimethyl-propyl)-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-5-yl]thiazol-2-yl]propanoyl]hexahydropyridazine-3-carboxylate (compound E8, 200.0 mg) as yellow gum. MS calc'd 1001.4 (MH⁺), measured 1001.4 (MH⁺).

### Step 8: Preparation of (3S)-1-[(2S)-2-(tert-butoxycarbonylamino)-3-[4-[6-fluoro-3-(3-hydroxy-2,2-dimethyl-propyl)-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-5-yl]thiazol-2-yl]propanoyl]hexahydropyridazine-3-carboxylic acid (Compound E9)

To a mixture of methyl (3S)-1-[(2S)-2-(tert-butoxycarbonylamino)-3-[4-[6-fluoro-3-(3-hydroxy-2,2-dimethyl-propyl)-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-5-yl]thiazol-2-yl]propanoyl]hexahydropyridazine-3-carboxylate (compound E8, 200.0 mg, 0.2 mmol) in DCE (5 mL) was added Me₃SnOH (200.0 mg, 1.11 mmol). The mixture was stirred at 60° C. for 12 hrs. The reaction mixture was concentrated under vacuum to give a residue. EtOAc (10 mL) and water (10) mL) were added to the residue and the layers were separated. The aqueous phase was extracted with EtOAc (15 mL, twice). The combined organic layer was washed with brine (20 mL), dried over Na₂SO₄, filtered, and concentrated under vacuum to afford (3S)-1-[(2S)-2-(tert-butoxycarbonylamino)-3-[4-[6-fluoro-3-(3-hydroxy-2,2-dimethyl-propyl)-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-5-yl]thiazol-2-yl]propanoyl]hexahydropyridazine-3-carboxylic acid (compound E9, 188.0 mg) as a brown solid. MS calc'd 987.4 (MH⁺), measured 987.4 (MH⁺).

### Step 9: Preparation of tert-butyl N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (Compound E10)

To a mixture of (3S)-1-[(2S)-2-(tert-butoxycarbonylamino)-3-[4-[6-fluoro-3-(3-hydroxy-2,2-dimethyl-propyl)-(2M)-2-[2-[(1S)-1-methoxyethyl]-5-[4-(2,2,2-trifluoroethyl)piperazin-1-yl]-3-pyridyl]-1-(2,2,2-trifluoroethyl)indol-5-yl]thiazol-2-yl]propanoyl]hexahydropyridazine-3-carboxylic acid (compound E9, 188.0 mg, 0.19 mmol) in DCM (20 mL) were added DIEA (0.7 mL, 3.81 mmol). EDCI (550.0 mg, 2.87 mmol) and HOBt (65.0 mg, 0.48 mmol) at 0° C. After being stirred at 20° C. for 12 hrs, the reaction mixture was poured into water (20 mL) and extracted with EtOAc (20 mL, three times). The combined organic layer was washed with brine (30 mL), dried over Na₂SO₄, filtered and concentrated under vacuum to give a residue which was purified by column chromatography (EtOAc in PE: 50%-70%) to afford tert-butyl N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound E10, 110.0 mg) as a yellow solid. MS calc'd 969.4 (MH⁺), measured 969.5 (MH⁺).

### Step 10: Preparation of (7S,13S)-7-amino-24-fluoro-(20M)-20-[2-[(1S)-1-methoxyethyl]-5-[4-(2,2,2-trifluoroethyl)piperazin-1-yl]-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]-octacosa-1(25),2,5(28),19,22(26),23-hexaene-8,14-dione (Intermediate E)

To a solution of tert-butyl N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]carbamate (compound E10, 110.0 mg, 0.11 mmol) in DCM (1 mL) was added TFA (1.0 mL, 12.98 mmol). The mixture was stirred at 20° C. for 1 h. After the reaction was completed, the reaction mixture was concentrated under vacuum to give a residue. Sat. NaHCO₃ aq. (20 mL) was added and the mixture was extracted with EtOAc (15 mL, three times). The combined organic layer was washed with brine (20 mL), dried over anhydrous sodium sulfate, filtered and concentrated in vacuo to afford (7S,13S)-7-amino-24-fluoro-(20M)-20-[2-[(1S)-1-methoxyethyl]-5-[4-(2,2,2-trifluoroethyl)piperazin-1-yl]-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]-octacosa-1(25),2,5(28),19,22(26),23-hexaene-8,14-dione (Intermediate E, 98.0 mg) as a yellow solid. MS calc'd 869.4 (MH⁺), measured 869.2 (MH⁺).

### Intermediate F

### (7S,13S)-7-amino-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

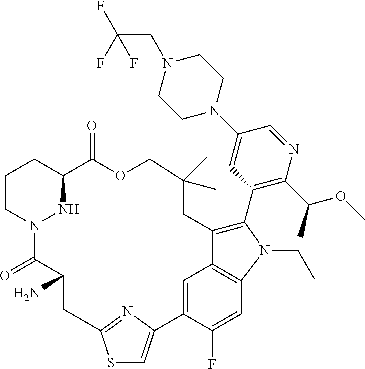

The title compound was prepared in analogy to the preparation of Intermediate E by using iodoethane instead of 2,2,2-trifluoroethyl trifluoromethanesulfonate.

### Intermediate G

### (7S,13S)-7-amino-21-ethyl-24-fluoro-(20M)-20-[2-[(1S)-1-methoxyethyl]-5-morpholino-3-pyridyl]-17,17-dimethyl-15-oxa-4-thia-9,21,27,28-tetrazapentacyclo-[17.5.2.1^{2,5}.1^{9,13}.0^{22,26}]octacosa-1(25),2,5(28),19,22(26),23-hexaene-8,14-dione

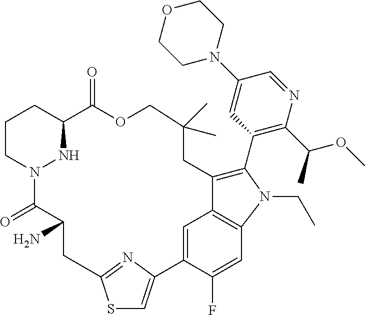

The title compound was prepared in analogy to the preparation of Intermediate E by using by using iodoethane and morpholine instead of 2,2,2-trifluoroethyl trifluoromethanesulfonate and 1-(2,2,2-trifluoroethyl)piperazine (compound E1).

### Intermediate H

### (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

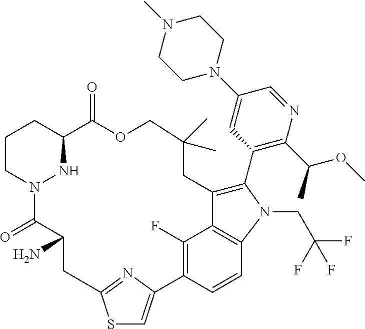

The title compound was prepared in analogy to the preparation of Intermediate C by using 5-bromo-4-fluoro-1H-indole instead of 5-bromo-6-fluoro-1H-indole (compound C2).

### Intermediate I

### (7S,13S)-7-amino-24-fluoro-(20M)-20-[2-[(1S)-1-methoxyethyl]-5-morpholino-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

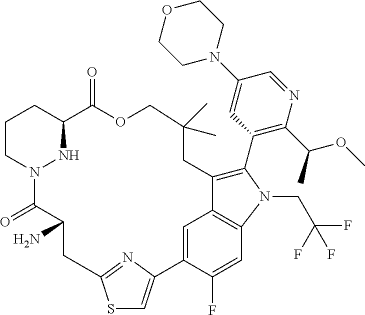

The title compound was prepared in analogy to the preparation of Intermediate E by using by using morpholine instead of 1-(2,2,2-trifluoroethyl)piperazine (compound E1).

### Example 1

### (1S,2S)—N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide

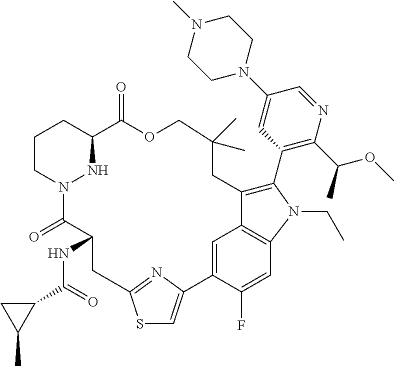

To a solution of (1S,2S)-2-methylcyclopropanecarboxylic acid (compound 1a, 64.3 mg, 0.64 mmol) in DMF (2 mL) were added DIEA (0.3 mL, 1.61 mmol), HATU (366.5 mg, 0.96 mmol) and (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D, 240.0 mg, 0.32 mmol) at 0° C. After being stirred at 20° C. for 16 hrs, the reaction mixture was poured into ice water (10 mL) and extracted with EA (20 mL, three times). The combined organic layer was washed with brine (20 mL), dried over Na₂SO₄ and concentrated under vacuum to give a residue. The resulting residue was purified by silica gel chromatography to afford (1S,2S)—N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide (Example 1, 156.41 mg) as a white solid. MS calc'd 829.5 (MH⁺), measured 829.5 (MH⁺). ¹H NMR (400 MHZ, METHANOL-d₄) δ=8.64 (d, J=7.6 Hz, 1H), 8.58 (d, J=8.4 Hz, 1H), 8.42 (d, J=2.9 Hz, 1H), 7.63 (d, J=2.4 Hz, 1H), 7.34 (d, J=2.8 Hz, 1H), 7.31 (d, J=12.7 Hz, 1H), 5.79-5.73 (m, 1H), 4.44 (br d, J=11.4 Hz, 1H), 4.28-4.13 (m, 4H), 3.77-3.68 (m, 2H), 3.41-3.37 (m, 4H), 3.28-3.13 (m, 2H), 3.02 (br d, J=13.6 Hz, 1H), 2.77-2.74 (m, 4H), 2.63 (br d, J=14.3 Hz, 1H), 2.44 (s, 3H), 2.21-2.13 (m, 1H), 1.97 (s, 2H), 1.68-1.58 (m, 1H), 1.51-1.47 (m, 1H), 1.42 (d, J=6.2 Hz, 2H), 1.37 (d, J=6.6 Hz, 4H), 1.13 (d, J=6.0 Hz, 3H), 1.08 (br dd, J=4.2, 8.6 Hz, 1H), 0.98 (t, J=7.0 Hz, 3H), 0.95-0.89 (m, 4H), 0.67-0.62 (m, 1H), 0.50 (s, 3H) ppm.

### Example 2

### (1R,5S)—N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-oxabicyclo[3.1.0]hexane-6-carboxamide

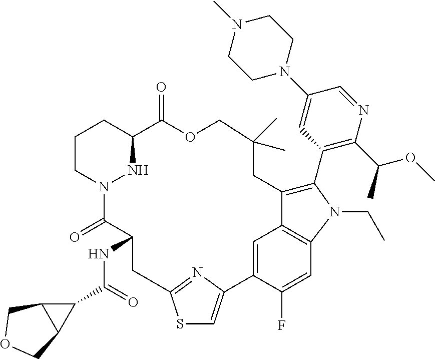

The title compound was prepared in analogy to the preparation of Example 1 by using (1S,5R)-3-oxabicyclo[3.1.0]hexane-6-carboxylic acid instead of (1S,2S)-2-methylcyclopropanecarboxylic acid (compound 1a). Example 2 (10.2 mg) was obtained as a white solid. MS calc'd 857.4 (MH⁺), measured 857.1 (MH⁺). ¹H NMR (400 MHZ, ACETONITRILE-d₃) δ=8.61 (d, J=7.6 Hz, 1H), 8.41 (d, J=2.8 Hz, 1H), 7.62 (d, J=2.5 Hz, 1H), 7.33-7.28 (m, 1H), 7.18 (d, J=2.8 Hz, 1H), 7.04 (br d, J=9.5 Hz, 1H), 5.68 (br t, J=8.8 Hz, 1H), 4.41-4.30 (m, 2H), 4.20-4.05 (m, 4H), 3.88-3.83 (m, 2H), 3.71-3.64 (m, 4H), 3.37 (br d, J=14.9 Hz, 1H), 3.28-3.24 (m, 4H), 3.22-3.17 (m, 3H), 3.13 (br dd, J=9.1, 15.0 Hz, 1H), 2.91 (br d, J=14.9 Hz, 1H), 2.70 (dt, J=2.7, 12.9 Hz, 1H), 2.58 (br d, J=14.1 Hz, 1H), 2.50 (br t, J=4.8 Hz, 4H), 2.30-2.22 (m, 4H), 1.78-1.72 (m, 1H), 1.57-1.50 (m, 2H), 1.36 (d, J=6.1 Hz, 3H), 1.27 (br s, 1H), 0.96 (br t, J=7.1 Hz, 3H), 0.88 (s, 3H), 0.47 (s, 3H) ppm.

### Example 3

### (1S,2S)-2-(difluoromethyl)-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]cyclopropanecarboxamide

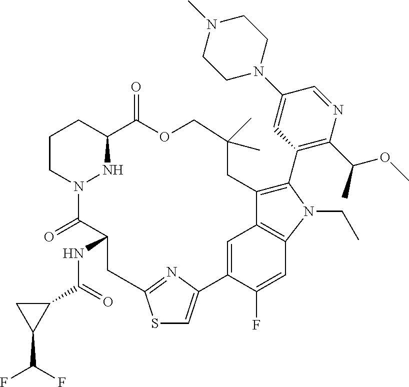

The title compound was prepared in analogy to the preparation of Example 1 by using (1S,2S)-2-(difluoromethyl)cyclopropanecarboxylic acid instead of (1S,2S)-2-methylcyclopropanecarboxylic acid (compound 1a). Example 3 (31.4 mg) was obtained as a yellow solid. MS calc'd 865.4 (MH⁺), measured 865.1 (MH⁺). ¹H NMR (400 MHZ, CHLOROFORM-d) δ=8.76 (br d, J=2.6 Hz, 1H), 8.66 (d, J=7.6 Hz, 1H), 7.63 (d, J=2.1 Hz, 1H), 7.30 (br d, J=2.4 Hz, 1H), 7.12 (d, J=12.3 Hz, 1H), 6.78 (br d, J=9.9 Hz, 1H), 6.00-5.92 (m, 1H), 4.66-4.53 (m, 1H), 4.36 (q, J=6.2 Hz, 1H), 4.31-4.23 (m, 1H), 4.19-4.04 (m, 2H), 3.89-3.86 (m, 1H), 3.78-3.66 (m, 4H), 3.47-3.44 (m, 1H), 3.41 (s, 3H), 3.24-3.05 (m, 6H), 2.93 (s, 3H), 2.89-2.80 (m, 1H), 2.78-2.70 (m, 1H), 2.53-2.39 (m, 1H), 2.29-2.18 (m, 1H), 2.08-1.66 (m, 5H), 1.53-1.45 (m, 3H), 1.19-1.14 (m, 2H), 1.37-1.08 (m, 2H), 1.02-0.87 (m, 6H), 0.50 (s, 3H) ppm.

### Example 4

### (1S,2S)—N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide

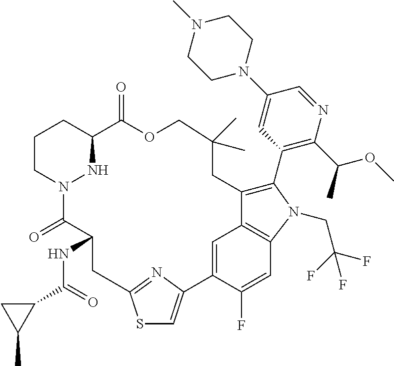

The title compound was prepared in analogy to the preparation of Example 1 by using (7S,13S)-7-amino-24-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate C) instead of (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate D). Example 4 (15.7 mg) was obtained as a white solid. MS calc'd 883.5 (MH⁺), measured 883.1 (MH⁺). ¹H NMR (400 MHZ, Methanol-d₄) δ=8.67 (d, J=7.6 Hz, 1H), 8.50 (d, J=2.8 Hz, 1H), 7.70 (d, J=2.0 Hz, 1H), 7.54-7.49 (m, 1H), 7.49-7.45 (m, 1H), 5.71 (d, J=9.2 Hz, 1H), 5.20-5.14 (m, 1H), 4.47-4.41 (m, 1H), 4.24-4.18 (m, 2H), 4.09-3.94 (m, 2H), 3.80-3.61 (, 5H), 3.49-3.43 (m, 2H), 3.35 (s, 3H), 3.17-3.12 (m, 2H), 3.00 (s, 3H), 2.83-2.76 (m, 1H), 2.57 (d, J=14.4 Hz, 1H), 2.23-2.16 (m, 1H), 1.99-1.93 (m, 1H), 1.84-1.77 (m, 1H), 1.68-1.61 (m, 1H), 1.52-1.48 (m, 1H), 1.47-1.41 (m, 4H), 1.27-1.22 (m, 1H), 1.13 (d, J=6.0 Hz, 3H), 1.11-1.05 (m, 2H), 0.96 (s, 3H), 0.90-0.77 (m, 1H), 0.68-0.62 (m, 1H), 0.44 (s, 3H).

### Example 5 and Example 6

### (1S,2S)-2-cyano-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]cyclopropanecarboxamide and (1R,2R)-2-cyano-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]cyclopropanecarboxamide

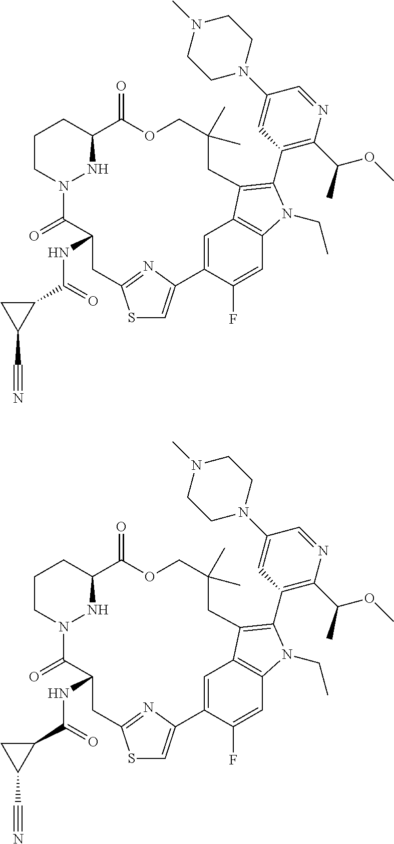

The compounds were prepared according to the following scheme:

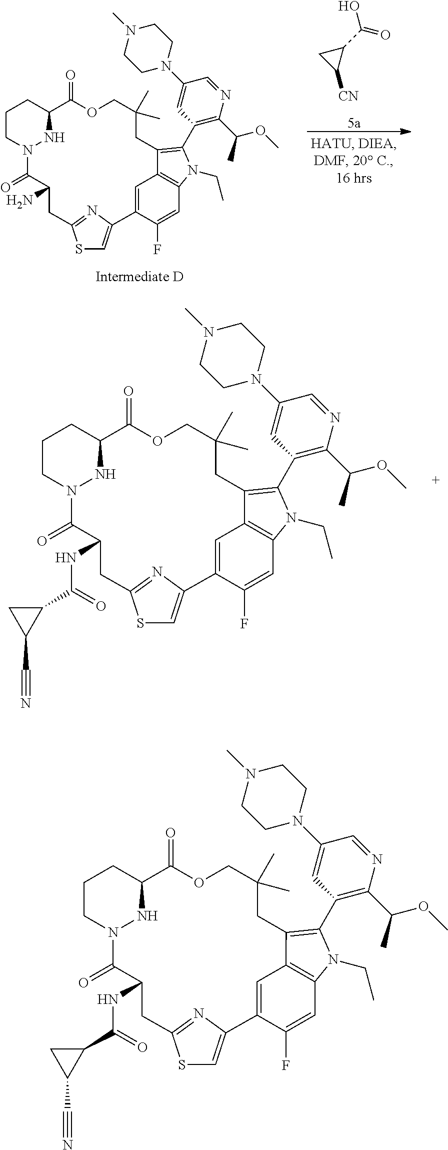

### Step 1: Preparation of (1S,2S)-2-cyano-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]cyclopropanecarboxamide and (1R,2R)-2-cyano-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]cyclopropanecarboxamide

To a solution of trans-2-Cyanocyclopropanecarboxylic acid (compound 5a, 11.2 mg, 0.1 mmol) in DMF (0.5 mL) were added DIEA (0.1 mL, 0.33 mmol), HATU (76.4 mg, 0.2 mmol) and (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate D, 50.0 mg, 0.07 mmol) at 0° C. After being stirred at 20° C. for 16 hrs, the reaction mixture was prepared by prep-HPLC and SFC to afford Example 5 (13.4 mg, faster eluted) as a white solid and Example 6 (9.9 mg, slower eluted) as a white solid.

Example 5: MS calc'd 840.4 (MH⁺), measured 840.1 (MH⁺). ¹H NMR (400 MHZ, CHLOROFORM-d) δ=8.63 (d, J=7.3 Hz, 1H), 8.49 (d, J=2.8 Hz, 1H), 7.59 (d, J=2.0 Hz, 1H), 7.12-7.07 (m, 2H), 6.88 (br d, J=8.4 Hz, 1H), 5.92 (t, J=8.9 Hz, 1H), 4.60 (br dd, J=2.4, 12.5 Hz, 1H), 4.30-4.19 (m, 3H), 4.08-3.99 (m, 2H), 3.85 (br d, J=11.1 Hz, 1H), 3.74-3.70 (m, 1H), 3.49 (br d, J=3.4 Hz, 4H), 3.36 (s, 3H), 3.20-3.13 (m, 2H), 2.76-2.68 (m, 4H), 2.63 (s, 1H), 2.46 (br d, J=14.2 Hz, 1H), 2.25-2.20 (m, 2H), 2.01 (s, 1H), 1.95-1.90 (m, 2H), 1.58 (td, J=5.0, 9.8 Hz, 2H), 1.47-1.42 (m, 5H), 1.30-1.22 (m, 4H), 0.97 (t, J=7.1 Hz, 3H), 0.91 (s, 2H), 0.47 (s, 3H) ppm.

Example 6: MS calc'd 840.4 (MH⁺), measured 840.1 (MH⁺). ¹H NMR (400 MHZ, CHLOROFORM-d) δ=8.64 (d, J=7.6 Hz, 1H), 8.51 (d, J=2.9 Hz, 1H), 7.63 (d, J=2.2 Hz, 1H), 7.13-7.07 (m, 2H), 6.88 (d, J=9.5 Hz, 1H), 5.95 (t, J=8.9 Hz, 1H), 4.65-4.57 (m, 1H), 4.32-4.21 (m, 3H), 4.12-3.96 (m, 2H), 3.86 (d, J=10.8 Hz, 1H), 3.75 (d, J=11.1 Hz, 1H), 3.48 (br d, J=14.7 Hz, 1H), 3.37 (s, 3H), 3.34-3.29 (m, 4H), 3.19 (dd, J=8.8, 15.0 Hz, 1H), 3.05 (br d, J=14.2 Hz, 1H), 2.76-2.62 (m, 5H), 2.52 (br d, J=14.3 Hz, 1H), 2.41 (s, 3H), 2.27-2.19 (m, 2H), 2.04-2.00 (m, 2H), 1.48-1.43 (m, 4H), 1.32-1.23 (m, 2H), 1.00 (t, J=7.1 Hz, 3H), 0.92 (s, 4H), 0.49 (s, 3H) ppm.

### Example 7

### (1S,2S)—N-[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide

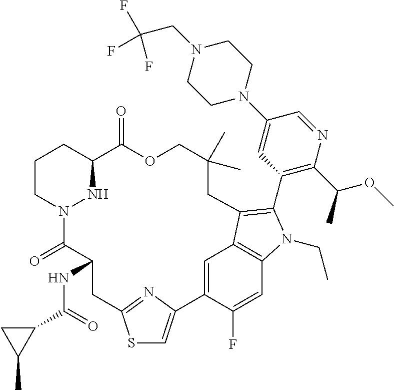

The title compound was prepared in analogy to the preparation of Example 1 by using (7S,13S)-7-amino-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate F) instead of (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28), 19,22(26),23-hexaene-8,14-dione (Intermediate D). Example 7 (12.5 mg) was obtained as a yellow solid. MS calc'd 897.4 (MH⁺), measured 897.6 (MH⁺). ¹H NMR (400 MHZ, Methanol-d₄) δ=8.68 (d, J=7.6 Hz, 1H), 8.38 (d, J=2.8 Hz, 1H), 7.78 (d, J=2.4 Hz, 1H), 7.65 (d, J=2.4 Hz, 1H), 7.35 (d, J=12.8 Hz, 1H), 5.81 (d, J=8.8 Hz, 1H), 4.48-4.40 (m, 1H), 4.38-4.31 (m, 1H), 4.30-4.19 (m, 1H), 4.16-4.01 (m, 2H), 3.81-3.68 (m, 2H), 3.49-3.44 (m, 4H), 3.43-3.40 (m, 1H), 3.37 (s, 3H), 3.21-3.12 (m, 3H), 3.04-3.95 (m, 1H), 2.92-2.86 (m, 4H), 2.81-2.70 (m, 2H), 2.21-2.11 (m, 1H), 1.99-1.89 (m, 1H), 1.84-1.70 (m, 1H), 1.67-1.57 (m, 1H), 1.51-1.46 (m, 1H), 1.44 (d, J=6.4 Hz, 3H), 1.25-1.19 (m, 1H), 1.15-1.10 (m, 3H), 1.09-1.05 (m, 1H), 1.04-1.00 (m, 3H), 0.94 (s, 3H), 0.66-0.61 (m, 1H), 0.59 (s, 3H) ppm.

### Example 8

### (1S,2S)—N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide

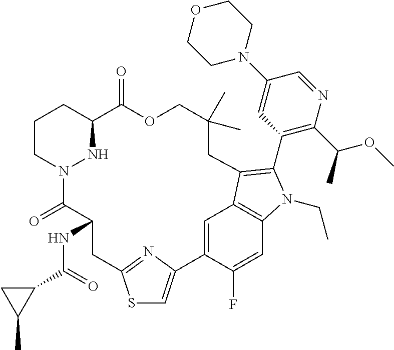

The title compound was prepared in analogy to the preparation of Example 1 by using (7S,13S)-7-amino-21-ethyl-24-fluoro-(20M)-20-[2-[(1S)-1-methoxyethyl]-5-morpholino-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate G) instead of (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate D). Example 8 (135.4 mg) was obtained as a light yellow solid. MS calc'd 816.4 (MH⁺), measured 816.1 (MH⁺). ¹H NMR (400 MHZ, Methanol-d₄) δ=8.70 (d, J=7.5 Hz, 1H), 8.37 (d, J=2.7 Hz, 1H), 7.95 (br s, 1H), 7.65 (d, J=2.3 Hz, 1H), 7.37 (d, J=12.6 Hz, 1H), 5.82 (br d, J=8.9 Hz, 1H), 4.47-4.38 (m, 2H), 4.31-4.23 (m, 1H), 4.15-4.10 (m, 1H), 4.05-3.98 (m, 1H), 3.88 (br t, J=4.7 Hz, 4H), 3.79 (br d, J=11.0 Hz, 1H), 3.71 (d, J=10.6 Hz, 1H), 3.47-3.38 (m, 10H), 3.00 (br d, J=15.0 Hz, 1H), 2.84-2.72 (m, 2H), 2.19-2.12 (m, 1H), 1.97-1.90 (m, 1H), 1.64 (br d, J=2.7 Hz, 1H), 1.50-1.45 (m, 4H), 1.23 (br dd, J=4.2, 9.5 Hz, 2H), 1.12 (d, J=5.9 Hz, 3H), 1.05 (q, J=6.8 Hz, 4H), 0.96 (s, 3H), 0.62 (s, 3H) ppm.

### Example 9

### (1S,2S)—N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide

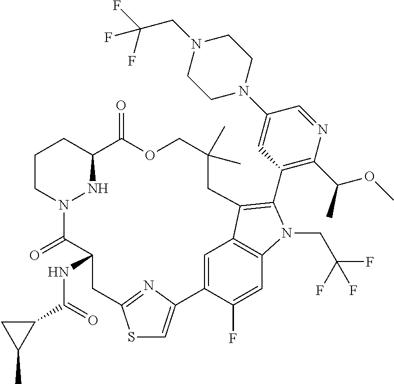

The title compound was prepared in analogy to the preparation of Example 1 by using (7S,13S)-7-amino-24-fluoro-(20M)-20-[2-[(1S)-1-methoxyethyl]-5-[4-(2,2,2-trifluoroethyl)piperazin-1-yl]-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate E) instead of (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate D). Example 9 (27.7 mg) was obtained as an off-white solid. MS calc'd 951.4 (MH⁺), measured 951.2 (MH⁺). ¹H NMR (400 MHZ, Methanol-d₄) δ=8.69 (d, J=7.6 Hz, 1H), 8.42 (d, J=2.8 Hz, 1H), 7.69 (d, J=2.4 Hz, 1H), 7.56-7.43 (m, 2H), 5.73 (d, J=8.8 Hz, 1H), 5.24-5.08 (m, 1H), 4.83-4.78 (m, 1H), 4.49-4.39 (m, 1H), 4.26-4.15 (m, 2H), 3.82-3.65 (m, 2H), 3.48-3.38 (m, 5H), 3.36 (s, 3H), 3.30-3.25 (m, 1H), 3.20-3.09 (m, 3H), 2.88 (t, J=4.8 Hz, 4H), 2.84-2.75 (m, 1H), 2.67-2.59 (m, 1H), 2.26-2.15 (m, 1H), 1.99-1.91 (m, 1H), 1.87-1.74 (m, 1H), 1.70-1.56 (m, 1H), 1.52-1.47 (m, 1H), 1.45 (d, J=6.0 Hz, 3H), 1.28-1.19 (m, 1H), 1.15-1.10 (m, 3H), 1.10-1.05 (m, 1H), 0.97 (s, 3H), 0.71-0.59 (m, 1H), 0.49 (s, 3H) ppm.

### Example 10

### (1S,2S)—N-[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide

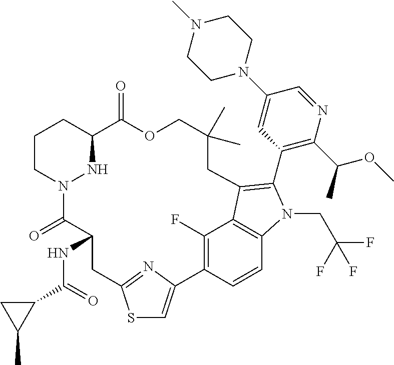

The title compound was prepared in analogy to the preparation of Example 1 by using (7S,13S)-7-amino-25-fluoro-(20M)-20-[2-[(1S)-1-methoxyethyl]-5-(4-methylpiperazin-1-yl)-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate H) instead of (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28), 19,22(26),23-hexaene-8,14-dione (Intermediate D). Example 10 (11.2 mg) was obtained as a white solid. MS calc'd 883.4 (MH⁺), measured 883.5 (MH⁺). ¹H NMR (400 MHZ, Methanol-d₄) δ=8.50 (d, J=2.8 Hz, 1H), 7.52-7.37 (m, 4H), 6.03-5.88 (m, 1H), 5.19-5.07 (m, 1H), 4.80-4.76 (m, 2H), 4.44-4.34 (m, 2H), 4.12-3.96 (m, 3H), 3.72-3.47 (m, 6H), 3.41-3.33 (m, 2H), 3.28-3.25 (m, 2H), 3.19-3.05 (m, 5H), 2.99 (s, 4H), 2.59-2.52 (m, 1H), 1.66-1.53 (m, 1H), 1.44 (d, J=6.0 Hz, 3H), 1.39-1.29 (m, 2H), 1.26-1.17 (m, 2H), 1.10-1.05 (m, 3H), 1.05-1.01 (m, 1H), 0.80 (s, 3H), 0.65-0.52 (m, 3H) ppm.

### Example 11

### (1S,2S)-2-(difluoromethyl)-N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]cyclopropanecarboxamide

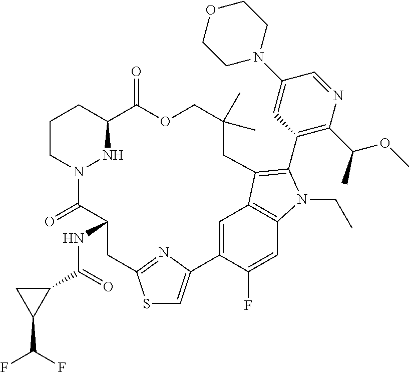

The title compound was prepared in analogy to the preparation of Example 1 by using (7S,13S)-7-amino-21-ethyl-24-fluoro-(20M)-20-[2-[(1S)-1-methoxyethyl]-5-morpholino-3-pyridyl]-17,17-dimethyl-15-oxa-4-thia-9,21,27,28-tetrazapentacyclo-[17.5.2.1^{2,5}.1^{9,13}.0^{22,26}]octacosa-1(25),2,5(28),19,22(26),23-hexaene-8,14-dione (Intermediate G) and (1S,2S)-2-(difluoromethyl)cyclopropanecarboxylic acid instead of (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (Intermediate D) and (1S,2 S)-2-methylcyclopropanecarboxylic acid (compound 1a). Example 11 (33.2 mg) was obtained as a yellow solid. MS calc'd 852.4 (MH⁺), measured 852.2 (MH⁺). ¹H NMR (400 MHZ, CHLOROFORM-d) δ=8.91 (br s, 1H), 8.66 (d, J=7.6 Hz, 1H), 7.62 (d, J=2.2 Hz, 1H), 7.41 (s, 1H), 7.13 (s, 1H), 7.10 (s, 1H), 6.75 (br d, J=8.9 Hz, 1H), 5.99-5.93 (m, 1H), 5.83 (d, J=3.1 Hz, 1H), 4.60 (br d, J=11.9 Hz, 1H), 4.38 (br d, J=6.4 Hz, 1H), 4.21 (br d, J=9.2 Hz, 1H), 4.13-4.06 (m, 2H), 3.87 (br d, J=11.4 Hz, 2H), 3.79-3.65 (m, 2H), 3.45 (br d, J=15.2 Hz, 1H), 3.41 (s, 2H), 3.39-3.35 (m, 4H), 3.17 (br dd, J=8.6, 15.0 Hz, 2H), 3.10 (br d, J=11.6 Hz, 1H), 2.80-2.63 (m, 2H), 2.50 (br d, J=13.9 Hz, 1H), 2.20 (br d, J=10.9 Hz, 1H), 1.99 (br d, J=17.9 Hz, 1H), 1.87-1.78 (m, 2H), 1.67-1.59 (m, 1H), 1.53 (d, J=6.4 Hz, 3H), 1.33-1.21 (m, 3H), 1.18-1.12 (m, 1H), 1.01 (br t, J=7.0 Hz, 3H), 0.94 (s, 3H), 0.53 (s, 3H) ppm.

### Example 12

### (1S,2S)—N-[(7S,13S)-24-fluoro-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide

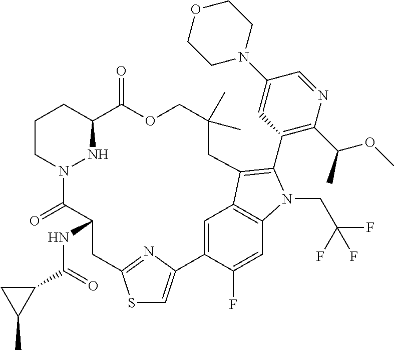

The title compound was prepared in analogy to the preparation of Example 1 by using (7S,13S)-7-amino-24-fluoro-(20M)-20-[2-[(1S)-1-methoxyethyl]-5-morpholino-3-pyridyl]-17,17-dimethyl-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate I) instead of (7S,13S)-7-amino-21-ethyl-24-fluoro-(20M)-20-[2-[(1S)-1-methoxyethyl]-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28), 19,22(26),23-hexaene-8,14-dione (Intermediate D). Example 12 (150 mg) was obtained as a white solid. MS calc'd 870.4 (MH⁺), measured 870.5 (MH⁺). ¹H NMR (400 MHZ, Methanol-d₄) δ=8.65-8.63 (d, J=7.6 Hz, 1H), 8.41-8.40 (d, J=2.8 Hz, 1H), 7.70-7.65 (d, J=2.4 Hz, 1H), 7.50-7.40 (d, J=12.4 Hz, 1H), 7.31-7.28 (d, J=2.4 Hz, 1H), 5.76-5.66 (d, J=8.0 Hz, 1H), 5.18-5.05 (m, 1H), 4.96-4.88 (m, 1H), 4.47-4.39 (m, 1H), 4.23-4.10 (m, 2H), 3.90-3.83 (t, J=4.8 Hz, J=9.6 Hz, 4H), 3.80-3.75 (d, J=10.8 Hz, 1H), 3.71-3.63 (m, 1H), 3.49-3.42 (m, 1H), 3.30-3.29 (m, 3H), 3.28-3.21 (m, 4H), 3.14-3.07 (d, J=14.4 Hz, 1H), 2.86-2.77 (m, 1H), 2.63-2.55 (d, J=14.4 Hz, 1H), 2.23-2.14 (m, 1H), 1.98-1.89 (m, 1H), 1.84-1.72 (m, 1H), 1.65-1.56 (m, 1H), 1.52-1.46 (m, 1H), 1.45-1.40 (d, J=6.4 Hz, 3H), 1.30-1.19 (m, 2H), 1.13-1.06 (m, 4H), 0.95 (s, 3H), 0.69-0.58 (m, 1H), 0.48-0.39 (s, 3H) ppm.

### Biological Example

Compound A122 (page 70 of Table, 1a) from WO2022060836 was cited as reference compound for this invention.

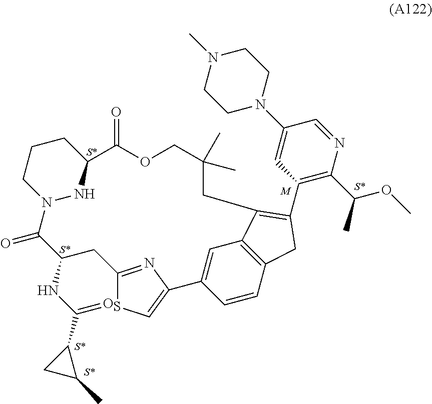

### Example 13

### Cell Viability Assay

The purpose of this cellular assay was to determine the effects of test compounds on the proliferation of human cancer cell lines NCI-H358 (ATCC-CRL5807) cells, AGS (ATCC-CRL-1739) cells, SW620 (ATCC-CCL-227) over a 3-day treatment period by quantifying the amount of NADPH present at endpoint using Cell Counting Kit-8.

Cells were seeded at 5,000 cells/well (NCI-H358), 2,000 cells/well (AGS) 2,000 cells/well (SW620) in 96-well assay plates (Corning-3699) and incubated overnight. On the day of the assay, diluted compounds were then added in a final concentration of 0.5% DMSO. After 72 hrs incubation, a tenth of the volume of cell counting kit 8 (Dnjindo-CK04) was added into each well. Read the signal (OD450 minus OD650) using EnVision after 2 hrs incubation. IC₅₀ was determined by fitting a 4-parameter sigmoidal concentration response model.

TABLE 1
Activity of Examples and Compounds of present
invention in KRAS Cell viability assay

| Example | G12C IC50 (μM) | G12D IC50 (μM) | G12V IC50 (μM) |
|---|---|---|---|
| A122 | <0.001 | 0.001 | <0.001 |
| Example 1 | 0.018 | 4.630 | 0.009 |
| Example 2 | 0.011 | 0.059 | 0.005 |
| Example 3 | 0.0116 | 0.197 | 0.008 |
| Example 4 | 0.019 | 1.265 | 0.013 |
| Example 5 | 0.023 | 0.089 | 0.012 |
| Example 6 | 0.079 | 0.316 | 0.035 |
| Example 7 | 0.074 | 0.389 | 0.027 |
| Example 8 | 0.016 | 1.493 | 0.013 |
| Example 9 | 0.224 | 0.704 | 0.040 |
| Example 10 | 0.012 | 0.013 | 0.003 |
| Example 11 | 0.005 | 0.111 | 0.007 |
| Example 12 | 0.011 | 0.244 | 0.016 |

### Example 14

KRAS-BRAF with CYPA (500 nM) Interaction Assay

In this example, TR-FRET was also used to measure the compound or compound-CYPA dependent disruption of the KRAS G12C-BRAF complex. This protocol was also used to measure disruption of KRAS G12D or KRAS G12V binding to BRAF by a compound of the invention, respectively. In assay buffer containing 25 mM HEPES PH=7.4 (4-(2-hydroxyethyl)-1-piperazineethanesulfonic acid, Thermo, 15630080), 0.002% Tween20, 0.1% BSA, 100 mM NaCl, 5 mM MgCl₂, 10 μM GMPPNP (Guanosine 5′-[β,γ-imido]triphosphate trisodium salt hydrate, Sigma, G0635), tagless CYPA, GMPPNP loaded 6His-KRAS proteins, and GST-BRAF^{RBD} were mixed in a well of a 384-well assay plate at final concentrations of 50 nM, 6.25 nM and 1 nM, respectively. Compound was present in plate wells as a 16-point 3-fold dilution series starting at a final concentration of 10 μM and incubated for 3 hours. A mixture of MAb Anti-6His-XL665 (Cisbio, 61HISXLB) and Mab anti-GST-TB cryptate (Cisbio, 61GSTTLB) was then added at a final concentration of 6.67 nM and 0.21 nM, respectively, and the plate was incubated for an additional 1.5 hours. TR-FRET signal was read on a PHERstar FSX microplate reader (Ex320 nm, Em 665/615 nm). Compounds that facilitate disruption of the KRAS-BRAF complex were identified as those eliciting a decrease in the TR-FRET ratio relative to DMSO control wells.

TABLE 2
Activity of Examples and Compounds of present invention
in KRAS-BRAF with CYPA (500 nM) interaction assay

| Example | G12C IC₅₀ (μM) | G12D IC₅₀ (μM) | G12V IC₅₀ (μM) |
|---|---|---|---|
| A122 | 0.013 | 0.124 | 0.025 |
| Example 1 | 0.108 | 0.915 | 0.211 |
| Example 2 | 0.134 | 0.957 | 0.174 |
| Example 3 | 0.196 | 1.715 | 0.243 |
| Example 4 | 0.036 | 0.544 | 0.095 |
| Example 5 | 0.194 | 0.862 | 0.258 |
| Example 6 | 0.154 | 0.701 | 0.273 |
| Example 7 | 0.192 | >10 | 0.697 |
| Example 8 | 0.160 | 1.674 | 0.143 |
| Example 9 | 0.090 | 0.660 | 0.171 |
| Example 10 | 0.009 | 0.020 | 0.013 |

### Example 15

### PERK Inhibition Assay

This assay is to measure the ability of test compounds in inhibiting the phosphorylation of ERK, the downstream signaling of KRAS G12C in NCI-H358 cells, KRAS G12D in AGS cells, and KRAS G12V in SW620. NCI-H358 (ATCC-CRL5807) cells, AGS (ATCC-CRL-1739) cells, SW620 (ATCC-CCL-227) cells were all grown and maintained using RPMI-1640 medium (Thermo Fisher Scientific) with 10% fetal bovine serum and 1% penicillin/streptomycin. On the day prior to compound addition, cells were plated in tissue culture-treated 96 well plates (Corning-3699) at a density of 30,000 cell/well, 20,000 cell/well, 30,000 cell/well for NCI-H358, AGS and SW620 respectively, and allowed for attachment overnight. Diluted compounds were then added in a final concentration of 0.5% DMSO. After 4 hours of incubation, the medium was removed, 100 μL of 4% formaldehyde was added, and the assay plates were incubated at room temperature for 20 minutes. The plates were then washed once with phosphate buffered saline (PBS), and permeabilized with 100 μL of chilled methanol for 10 minutes. Non-specific antibody binding to the plates was blocked using 50 μL 1×BSA blocking buffer (Thermo-37520, 10-fold dilution by Phosphate-Buffered Saline Tween (PBST) for at least 1 hour at room temperature.

The amount of phosphor-ERK was determined using an antibody specific for phosphorylated form of ERK. Primary antibody (pERK, CST-4370, Cell Signaling Technology) was diluted 1:300 in blocking buffer, with 50 μL aliquoted to each well, and incubated overnight at 4° C. Cells was washed five times for 5 minutes with PBST. Secondary antibody (HRP-linked anti-rabbit IgG, CST-7074, Cell Signaling Technology) was diluted 1:1000 in blocking buffer, and 50 μL was added to each well and incubated 1-2 hrs at room temperature. Cells was washed 5 times for 5 minutes with PBST, 100 μL TMB ELISA substrate (abcam-ab171523) were added and gently shake for 20 minutes. 50 μL stop solution (abcam-ab171529) were added, and then read the signal (OD450) by EnVision.

IC₅₀ was determined by fitting a 4-parameter sigmoidal concentration response model.

TABLE 3
Activity of Examples and Compounds of present
invention in KRAS pERK inhibition assay

| Example | G12C IC₅₀ (μM) | G12D IC₅₀ (μM) | G12V IC₅₀ (μM) |
|---|---|---|---|
| A122 | 0.002 | 0.001 | <0.001 |
| Example 1 | 0.011 | 0.034 | 0.004 |
| Example 2 | 0.009 | 0.018 | 0.003 |
| Example 3 | 0.011 | 0.029 | 0.005 |
| Example 4 | 0.02 | 0.047 | 0.007 |
| Example 5 | 0.057 | 0.056 | 0.013 |
| Example 6 | 0.079 | 0.116 | 0.025 |
| Example 7 | 0.071 | 0.115 | 0.02 |
| Example 8 | 0.023 | 0.056 | 0.008 |
| Example 9 | 0.047 | 0.161 | 0.022 |
| Example 10 | 0.017 | 0.011 | 0.002 |
| Example 11 | 0.021 | 0.028 | 0.007 |
| Example 12 | 0.033 | 0.053 | 0.009 |

1. A compound of formula (I), 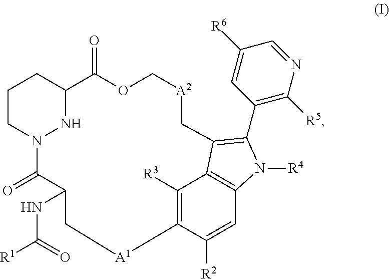
wherein
R¹ is 3-oxabicyclo[3.1.0]hexanyl, C₁₋₆alkylC₃₋₇cycloalkyl, cyanoC₃₋₇cycloalkyl or haloC₁₋₆alkylC₃₋₇cycloalkyl;
R² is H or halogen;
R³ is H or halogen;
R⁴ is C₁₋₆alkyl or haloC₁₋₆alkyl;
R⁵ is C₁₋₆alkoxyC₁₋₆alkyl;
R⁶ is morpholinyl, (haloC₁₋₆alkyl)piperazinyl or C₁₋₆alkylpiperazinyl;
A¹ is thiazolylene;
A² is C₁₋₆alkylene;
with the proviso that R² and R³ are not H simultaneously;
or a pharmaceutically acceptable salt thereof.

2. The compound or pharmaceutically acceptable salt of claim 1, wherein the compound is a compound of formula (Ia), 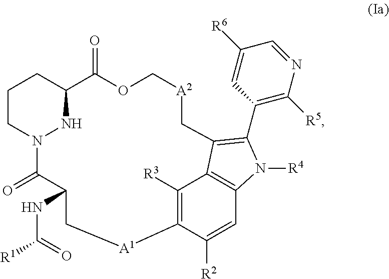
wherein
R¹ is 3-oxabicyclo[3.1.0]hexanyl, C₁₋₆alkylC₃₋₇cycloalkyl, cyanoC₃₋₇cycloalkyl or haloC₁₋₆alkylC₃₋₇cycloalkyl;
R² is H or halogen;
R³ is H or halogen;
R⁴ is C₁₋₆alkyl or haloC₁₋₆alkyl;
R⁵ is C₁₋₆alkoxyC₁₋₆alkyl;
R⁶ is morpholinyl, (haloC₁₋₆alkyl)piperazinyl or C₁₋₆alkylpiperazinyl;
A¹ is thiazolylene; and
A² is C₁₋₆alkylene;
with the proviso that R² and R³ are not H simultaneously.

3. The compound or pharmaceutically acceptable salt according to claim 1, wherein R¹ is C₁₋₆alkylC₃₋₇cycloalkyl or haloC₁₋₆alkylC₃₋₇cycloalkyl.

4. The compound or pharmaceutically acceptable salt according to claim 1, wherein R¹ is methylcyclopropyl or (difluoromethyl)cyclopropyl.

5. The compound or pharmaceutically acceptable salt according to claim 1, wherein R² is H or fluoro.

6. The compound or pharmaceutically acceptable salt according to claim 1, wherein R³ is H or fluoro.

7. The compound or pharmaceutically acceptable salt according to claim 1, wherein R⁴ is ethyl or 2,2,2-trifluoroethyl.

8. The compound or pharmaceutically acceptable salt according to claim 1, wherein R⁵ is 1-methoxyethyl.

9. The compound or pharmaceutically acceptable salt according to claim 1, wherein R⁶ is morpholinyl or C₁₋₆alkylpiperazinyl.

10. The compound or pharmaceutically acceptable salt according to claim 1, wherein R⁶ is morpholinyl or 4-methylpiperazin-1-yl.

11. The compound or pharmaceutically acceptable salt according to claim 1, wherein A¹ is 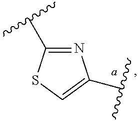 [SMILES: *c1csc(*)n1]
wherein bond “a” connects to indole ring.

12. The compound or pharmaceutically acceptable salt according to claim 1, wherein A² is dimethylmethylene.

13. The compound or pharmaceutically acceptable salt according to claim 1, wherein
R¹ is C₁₋₆alkylC₃₋₇cycloalkyl or haloC₁₋₆alkylC₃₋₇cycloalkyl;
R² is H or halogen;
R³ is H or halogen;
R⁴ is C₁₋₆alkyl or haloC₁₋₆alkyl;
R⁵ is C₁₋₆alkoxyC₁₋₆alkyl;
R⁶ is morpholinyl or C₁₋₆alkylpiperazinyl;
A¹ is 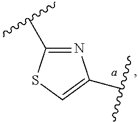 [SMILES: *c1csc(*)n1]
wherein bond “a” connects to indole ring; and
A² is C₁₋₆alkylene;
with the proviso that R² and R³ are not H simultaneously.

14. The compound or pharmaceutically acceptable salt according to claim 1, wherein
R¹ is 2-methylcyclopropyl or 2-(difluoromethyl)cyclopropyl;
R² is H or fluoro;
R³ is H or fluoro;
R⁴ is ethyl or 2,2,2-trifluoroethyl;
R⁵ is (1S)-1-methoxyethyl;
R⁶ is morpholinyl or 4-methylpiperazin-1-yl;
A¹ is 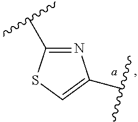 [SMILES: *c1csc(*)n1]
wherein bond “a” connects to indole ring; and
A² is dimethylmethylene;
with the proviso that R² and R³ are not H simultaneously.

15. The compound or pharmaceutically acceptable salt according to claim 1, wherein the compound is selected from:
(1S,2S)—N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28), 19,22(26),23-hexaen-7-yl]-2-methyl-cyclopropanecarboxamide;
(1R,5S)—N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-3-oxabicyclo[3.1.0]hexane-6-carboxamide;
(1S,2S)-2-(difluoromethyl)-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]cyclopropanecarboxamide;
(1S,2S)—N-[(7S,13S)-24-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide;
(1S,2S)-2-cyano-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]cyclopropanecarboxamide;
(1R,2R)-2-cyano-N-[(7S,13S)-21-ethyl-24-fluoro-(20M)-20-[2-[(1S)-1-methoxyethyl]-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]cyclopropanecarboxamide;
(1S,2S)—N-[(7S,13S)-21-ethyl-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide;
(1S,2S)—N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide;
(1S,2S)—N-[(7S,13S)-24-fluoro-(20M)-20-[2-[(1S)-1-methoxyethyl]-5-[4-(2,2,2-trifluoroethyl)piperazin-1-yl]-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide;
(1S,2S)—N-[(7S,13S)-25-fluoro-(20M)-20-[2-[(1S)-1-methoxyethyl]-5-(4-methylpiperazin-1-yl)-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide;
(1S,2S)-2-(difluoromethyl)-N-[(7S,13S)-21-ethyl-24-fluoro-(20M)-20-[2-[(1S)-1-methoxyethyl]-5-morpholino-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]cyclopropanecarboxamide; and
(1S,2S)—N-[(7S,13S)-24-fluoro-(20M)-20-[2-[(1S)-1-methoxyethyl]-5-morpholino-3-pyridyl]-17,17-dimethyl-8,14-dioxo-21-(2,2,2-trifluoroethyl)-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide.

16. A process for the preparation of a compound of formula (I) comprising:
a) coupling reaction between compound of formula (II), 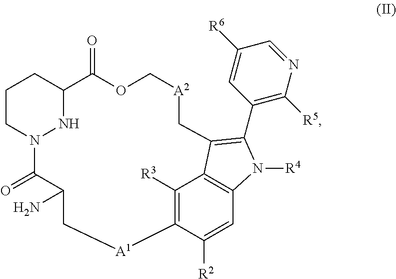
and acid (III), 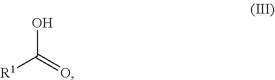
in the presence of a coupling reagent and a base to form the compound of formula (I);
wherein
R¹ is 3-oxabicyclo[3.1.0]hexanyl, C₁₋₆alkylC₃₋₇cycloalkyl, cyanoC₃₋₇cycloalkyl or haloC₁₋₆alkylC₃₋₇cycloalkyl;
R² is H or halogen;
R³ is H or halogen;
R⁴ is C₁₋₆alkyl or haloC₁₋₆alkyl;
R⁵ is C₁₋₆alkoxyC₁₋₆alkyl;
R⁶ is morpholinyl, (haloC₁₋₆alkyl)piperazinyl or C₁₋₆alkylpiperazinyl;
A¹ is thiazolylene; and
A² is C₁₋₆alkylene;
with the proviso that R² and R³ are not H simultaneously;
wherein the coupling reagent is T₃P, HATU, PyBOP or EDCI/HOBt; and the base is TEA, DIEPA or DMAP.

17. (canceled)

18. A pharmaceutical composition comprising the compound or pharmaceutically acceptable salt according to claim 1 and a pharmaceutically acceptable excipient.

19-26. (canceled)

27. A method for the treatment of a KRAS mutation driven cancer, wherein the cancer is selected from pancreatic adenocarcinoma, colorectal cancer and non-small cell lung cancer, which method comprises administering a therapeutically effective amount of the compound or therapeutically acceptable salt of claim 1.

28. (canceled)

29. (canceled)
